SEQUENCE LISTING (85 sequences)

SEQ ID NO: 1 - protein, 758 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 10 aa, Human
MAEPRQEFEV

SEQ ID NO: 3 - protein, 20 aa, Human
MAEPRQEFEVMEDHAGTYGL

SEQ ID NO: 4 - protein, 30 aa, Human
MAEPRQEFEVMEDHAGTYGLGDRKDQGGYT

SEQ ID NO: 5 - protein, 40 aa, Human
MAEPRQEFEVMEDHAGTYGLGDRKDQGGYTMHQDQEGDTD

SEQ ID NO: 6 - protein, 50 aa, Human
MAEPRQEFEVMEDHAGTYGLGDRKDQGGYTMHQDQEGDTDAGLKESPLQT

SEQ ID NO: 7 - protein, 60 aa, Human
MAEPRQEFEVMEDHAGTYGLGDRKDQGGYTMHQDQEGDTDAGLKESPLQTPTEDGSEEPG

SEQ ID NO: 8 - protein, 70 aa, Human
MAEPRQEFEVMEDHAGTYGLGDRKDQGGYTMHQDQEGDTDAGLKESPLQTPTEDGSEEPGSETSDAKSTP

SEQ ID NO: 9 - protein, 80 aa, Human
MAEPRQEFEVMEDHAGTYGLGDRKDQGGYTMHQDQEGDTDAGLKESPLQTPTEDGSEEPGSETSDAKSTPTAEDVTAPLV

SEQ ID NO: 10 - protein, 90 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAA

SEQ ID NO: 11 - protein, 100 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEG

SEQ ID NO: 12 - protein, 110 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGD

SEQ ID NO: 13 - protein, 120 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAG

SEQ ID NO: 14 - protein, 130 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGK

SEQ ID NO: 15 - protein, 140 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREP

SEQ ID NO: 16 - protein, 150 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLM

SEQ ID NO: 17 - protein, 160 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLP

SEQ ID NO: 18 - protein, 170 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQP

SEQ ID NO: 19 - protein, 180 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEG

SEQ ID NO: 20 - protein, 190 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKH

SEQ ID NO: 21 - protein, 200 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEG

SEQ ID NO: 22 - protein, 210 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKE

SEQ ID NO: 23 - protein, 220 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDE

SEQ ID NO: 24 - protein, 230 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQ

SEQ ID NO: 25 - protein, 240 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPA

SEQ ID NO: 26 - protein, 250 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAA

SEQ ID NO: 27 - protein, 260 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFP

SEQ ID NO: 28 - protein, 270 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVD

SEQ ID NO: 29 - protein, 280 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIP

SEQ ID NO: 30 - protein, 290 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVG

SEQ ID NO: 31 - protein, 300 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLE

SEQ ID NO: 32 - protein, 310 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPN

SEQ ID NO: 33 - protein, 320 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEE

SEQ ID NO: 34 - protein, 330 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGA

SEQ ID NO: 35 - protein, 340 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGP

SEQ ID NO: 36 - protein, 350 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEAD

SEQ ID NO: 37 - protein, 360 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPA

SEQ ID NO: 38 - protein, 370 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSR

SEQ ID NO: 39 - protein, 380 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVS

SEQ ID NO: 40 - protein, 390 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDD

SEQ ID NO: 41 - protein, 400 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSS

SEQ ID NO: 42 - protein, 410 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCL

SEQ ID NO: 43 - protein, 420 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSS

SEQ ID NO: 44 - protein, 430 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPA

SEQ ID NO: 45 - protein, 440 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPK

SEQ ID NO: 46 - protein, 450 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTG

SEQ ID NO: 47 - protein, 460 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLK

SEQ ID NO: 48 - protein, 470 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIAT

SEQ ID NO: 49 - protein, 480 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQK

SEQ ID NO: 50 - protein, 490 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPA

SEQ ID NO: 51 - protein, 500 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPP

SEQ ID NO: 52 - protein, 510 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGD

SEQ ID NO: 53 - protein, 520 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSP

SEQ ID NO: 54 - protein, 530 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTP

SEQ ID NO: 55 - protein, 540 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREP

SEQ ID NO: 56 - protein, 550 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPP

SEQ ID NO: 57 - protein, 560 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRL

SEQ ID NO: 58 - protein, 570 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDL

SEQ ID NO: 59 - protein, 580 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGST

SEQ ID NO: 60 - protein, 590 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGG

SEQ ID NO: 61 - protein, 600 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLD

SEQ ID NO: 62 - protein, 610 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGS

SEQ ID NO: 63 - protein, 620 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGG

SEQ ID NO: 64 - protein, 630 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPV

SEQ ID NO: 65 - protein, 640 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCG

SEQ ID NO: 66 - protein, 650 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPG

SEQ ID NO: 67 - protein, 660 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEK

SEQ ID NO: 68 - protein, 670 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSK

SEQ ID NO: 69 - protein, 680 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHV

SEQ ID NO: 70 - protein, 690 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIET

SEQ ID NO: 71 - protein, 700 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAK

SEQ ID NO: 72 - protein, 710 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIV

SEQ ID NO: 73 - protein, 720 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDT

SEQ ID NO: 74 - protein, 730 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSS

SEQ ID NO: 75 - protein, 740 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSP

SEQ ID NO: 76 - protein, 750 aa, Human
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVS

SEQ ID NO: 77 - protein, 18 aa, Human
QLATLADEVSASLAKQGL

SEQ ID NO: 78 - protein, 28 aa, Human
TGSIDMVDSPQLATLADEVSASLAKQGL

SEQ ID NO: 79 - protein, 30 aa, Human
SSTGSIDMVDSPQLATLADEVSASLAKQGL

SEQ ID NO: 80 - protein, 40 aa, Human
DTSPRHLSNVSSTGSIDMVDSPQLATLADEVSASLAKQGL

SEQ ID NO: 81 - protein, 10 aa, Human
YERKFWHGRH

SEQ ID NO: 82 - protein, 11 aa, Human
DGASYSWMYGA

SEQ ID NO: 83 - protein, 24 aa, Human
CQYRCFQVITNGIGLNLFKDPVAD

SEQ ID NO: 84 - protein, 24 aa, Human
CSFKCYSVVTNGLGINVFKDPVAD

SEQ ID NO: 85 - protein, 24 aa, Human
CQYRCFQIITNGIGLNLFKDPVAD

(19) United States
(12) Patent Application Publication
(10) Pub. No.: US 2015/0233904 A1
(43) Pub. Date: Aug. 20, 2015

(54) DETECTION OF NEUROLOGICAL DISEASES VIA MEASUREMENT OF NEUROMELANIN IN RECIRCULATING PHAGOCYTES

(71) Applicant: MSDX, INC., TUCSON, AZ (US)

(72) Inventor: Ramesh C. Nayak, Tucson, AZ (US)

(21) Appl. No.: 14/704,791

(22) Filed: May 5, 2015

Related U.S. Application Data
(63) Continuation-in-part of application No. PCT/US13/68465, filed on Nov. 5, 2013.
Application No. PCT/US13/68465 is a continuation-in-part of application No. 12/954,505, filed on Nov. 24, 2010.
(60) Provisional application No. 61/722,441, filed on Nov. 5, 2012.
(60) Provisional application No. 61/393,254, filed on Oct. 14, 2010.
(60) Provisional application No. 61/371,122, filed on Aug. 5, 2010.
(60) Provisional application No. 61/264,760, filed on Nov. 27, 2009.

(51) Int. Cl. G01N 33/53

(52) U.S. Cl. CPC G01N 33/5308; G01N 2800/2835

(57) ABSTRACT

Methods for detecting or monitoring neurological or neurodegenerative diseases such as Parkinson's disease via detection or measurement of central nervous system biomarkers within recirculating phagocytes after re-entry into the blood stream. The methods of the present invention may feature detecting neuromelanin in such recirculating phagocytes. For example, neuromelanin-binding peptides may be used to detect neuromelanin in recirculating phagocytes.

### CROSS REFERENCE

This application is a continuation-in-part of and claims priority to PCT Application No. PCT/US13/68465 filed on Nov. 5, 2013, which claims priority to U.S. Provisional Patent Application No. 61/722,441 filed on Nov. 5, 2012, the specification(s) of which is/are incorporated herein in their entirety by reference.

This application is a continuation-in-part of and claims priority to U.S. patent application Ser. No. 12/954,505 filed on Nov. 24, 2010, claims priority to U.S. Provisional Patent Application No. 61/264,760 filed Nov. 27, 2009, U.S. Provisional Patent Application No. 61/371,122 filed Aug. 5, 2010, and U.S. Provisional Patent Application No. 61/393,254 filed on Oct. 14, 2010, the specification(s) of which is/are incorporated herein in their entirety by reference.

### BACKGROUND OF THE INVENTION

In general, when tissue damage occurs, it incites inflammation, which usually aids in wound healing. For example, one of the normal functions of inflammation is to recruit phagocytes to clear away the cellular debris and prepare the injured site for repair and rebuilding. These phagocytes may be resident in the brain (e.g., dendritic cells, microglial cells) or recruited from the blood stream (e.g., monocytes). Cells that engulf debris are thought to enter the brain by crossing the blood-brain barrier but are not believed to return to the blood stream. For example, when phagocytes engulf tissue debris and exit the tissue, it is thought to be via the lymph notes.

We have surprisingly discovered that debris-laden phagocytes may re-enter the blood stream and if sufficient numbers are present, it may be possible to measure the debris that is inside the phagocytic cells. For example, we have found that the CNS antigens Tau and Hippocalcin like-1 are present in PBMC preparations at a frequency that is statistically different from apparently healthy controls. Examining the cargo (e.g., the debris that would only normally be found in nerve cells) of these recirculating phagocytes is termed “Window into the Brain.” This technique may provide close to real-time data on what is happening in the brain since that particular cargo may only be present in the recirculating phagocytes for a few days before it is completely digested. The present invention features methods of detecting various diseases by examining the debris present in such recirculating phagocytes.

The present invention also features methods for detecting Parkinson's disease by the detection of melanin (e.g., neuromelanin, e.g., neuromelanin from neurons of substantia nigra) and other neuronal antigens in recirculating phagocytes.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features a method of detecting Parkinson's disease in a mammal. In some embodiments, the method comprises detecting a level of a biomarker associated with Parkinson's disease in a first sample from outside a brain tissue of the mammal, the first sample comprising a first circulating phagocyte; and comparing the level of the biomarker in the first sample with a level of the biomarker in a second sample, the second sample being either (i) a control sample or (ii) a second sample from outside of a brain tissue, the second sample comprising a second circulating phagocyte, the second sample being collected prior to the first fluid sample, wherein if the level of the biomarker in the first sample is higher than that of the second sample then Parkinson's disease is detected.

In some embodiments, the sample is derived from blood, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, urine, the like, or a combination thereof. In some embodiments, the biomarker associated with Parkinson's disease comprises neuromelanin or a fragment thereof. In some embodiments, the circulating phagocyte includes a monocyte, a macrophage, a lymphocyte, or a combination thereof. In some embodiments, detecting the biomarker comprises subjecting the first sample and the second sample each to a peptide that binds to neuromelanin. In some embodiments, the peptide that binds to neuromelanin comprises 4B4 (SEQ ID NO:1A).

The present invention also features a kit for detecting Parkinson's disease, said kit comprising a 4B4 peptide (SEQ ID NO:1A), the 4B4 peptide is for detecting neuromelanin in a recirculating phagocyte. In some embodiments, the 4B4 peptide comprises a label. In some embodiments, the label comprises biotin.

The present invention also features the use of a system for detecting Parkinson's disease. In some embodiments, the system comprises a neuromelanin-binding peptide for binding to neuromelanin, the neuromelanin-binding peptide is incubated in a first sample comprising a first circulating phagocyte from outside of a brain tissue and a second sample comprising a control sample, wherein if the level of neuromelanin detected in the first sample via the neuromelanin-binding peptide is higher than the level of neuromelanin detected in the second sample via the neuromelanin-binding peptide then Parkinson's disease is detected. In some embodiments, the neuromelanin-binding peptide comprises 4B4 (SEQ ID NO:1A). In some embodiments, the first sample is derived from blood. In some embodiments, the first sample comprises PBMCs.

The present invention also features a system for detecting Parkinson's disease, wherein the system comprises a neuromelanin-binding peptide for binding to neuromelanin, the neuromelanin-binding peptide is incubated in a first sample comprising a first circulating phagocyte from outside of a brain tissue and a second sample comprising a control sample, wherein if the level of neuromelanin detected in the first sample via the neuromelanin-binding peptide is higher than the level of neuromelanin detected in the second sample via the neuromelanin-binding peptide then Parkinson's disease is detected. In some embodiments, the neuromelanin-binding peptide comprises 4B4 (SEQ ID NO:1A). In some embodiments, the first sample is derived from blood. In some embodiments, the first sample comprises PBMCs.

The present invention also features a method of determining status of Parkinson's disease. In some embodiments, method comprises detecting a level of a biomarker associated with Parkinson's disease in a first fluid sample from outside a brain tissue of the mammal, the first fluid sample comprising a first circulating phagocyte; comparing the level of the biomarker in the first sample with a level of the biomarker in a second sample, the second sample being either (i) a control sample or (ii) a second fluid sample from outside of a brain tissue, the second fluid sample comprising a second circulating phagocyte, the second fluid sample being collected prior to the first fluid sample.

In some embodiments, if the biomarker level in the first sample is the same as the level of the biomarker in the second sample or in the control sample, then Parkinson's disease activity is the same. In some embodiments, if the biomarker level in the first sample is higher than the level of the biomarker in the second sample or in the control sample, then Parkinson's disease activity is increased in the first sample. In some embodiments, if the biomarker level in the first sample is lower than the level of the biomarker in the second sample or in the control sample, then Parkinson's disease activity is decreased in the first sample.

In some embodiments, the sample is derived from blood, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, urine, the like, or a combination thereof. In some embodiments, the circulating phagocyte includes a monocyte, a macrophage, a lymphocyte, or a combination thereof. In some embodiments, the biomarker comprises neuromelanin or a fragment thereof.

The present invention also features a method of detecting neuromelanin. In some embodiments, the method comprises introducing a neuromelanin binding protein comprising a labeled 4B4 peptide (SEQ ID NO:1A) to a sample; and detecting the label on the 4B4 peptide. In some embodiments, the sample comprises a circulating phagocyte. In some embodiments, the sample comprises a circulating phagocyte derived from serum, plasma, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, or a combination thereof. In some embodiments, the label comprises an enzyme. In some embodiments, the label comprises biotin. In some embodiments, the enzyme comprises horseradish peroxidase.

The present invention also features a method of detecting Parkinson's disease in a patient. In some embodiments, the method comprises obtaining from a patient a fluid sample from outside of a brain tissue of the patient, the fluid sample comprises peripheral blood mononuclear cells (PBMCs); and detecting neuromelanin in the fluid sample, wherein when neuromelanin is detected then Parkinson's disease is detected in the patient. In some embodiments, the fluid sample comprises a circulating phagocyte. In some embodiments, the circulating phagocyte includes a monocyte, a macrophage, or a lymphocyte.

### BRIEF DESCRIPTION OF THE DRAWINGS

FIG. 1: Groups of plasma samples from multiple sclerosis (MS) patients that had low, medium or high levels of MSDx complex-1 were selected and then MMP-9 was measured. FIG. 1 shows the MMP-9 level was elevated in MS patients relative to normal subjects (p=0.01-0.006) and did not vary by MSDx complex-1 level in MS subjects (p=not significant). TIMP-1 levels were higher in subjects with low MSDx complex-1 levels (consistent with low proteolytic activity and potentially lower levels of leukocyte invasion and disease activity). MS subjects with high MSDX complex-1 level had lower levels of TIMP-1 (consistent with higher proteolytic activity and potentially higher levels of leukocyte invasion and disease activity; TIMP-1 in MSDx complex-1 high vs. Low p=0.0033).

FIG. 2: Image (a) on the left shows neuromelanin-containing dopaminergic neurons in the human substantia nigra revealed by the Masson-Fontana stain. Image (b) on the right shows neuromelanin-containing dopaminergic neurons in the human substantia nigra revealed by the 4B4 peptide binding (4B4 peptide binds to neuromelanin in substantia nigra tissue sections).

FIG. 3 shows the binding of the 4B4 peptide to neuromelanin in extracts of retinal pigment epithelium immobilized on ELISA plates in two-fold dilution series.

### DESCRIPTION OF PREFERRED EMBODIMENTS

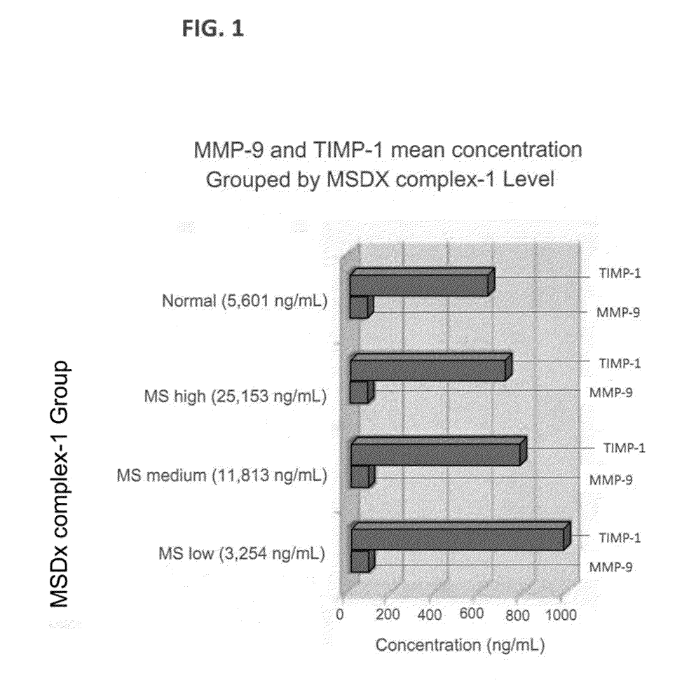

Referring now to FIG. 1-3, the present invention features the detection and/or the monitoring of diseases, e.g., neurodegenerative diseases, Parkinson's disease, etc., by analysis of phagocytosed central nervous system (CNS) debris within phagocytes that have re-entered the blood circulation (e.g., recirculating phagocytes).

### MSDX Complex-1

MSDx complex-1 comprises Fibrinogen, Fibulin-1 and Fibronectin. Fibronectin and Fibulin-1 are basement membrane proteins, suggesting that the circulating complex may be generated as a consequence of leukocyte transmigration into target tissues. Transmigration of leukocytes is mediated by the enzyme activity of matrix metalloproteinases (MMPs).

FIG. 1 shows that in plasma samples of Multiple Sclerosis patients, levels of MSDx complex-1 may be indirectly related to TIMP-1 levels (TIMPs are tissue inhibitors of metalloproteinases). For example, higher levels of TIMP-1 (a specific inhibitor of MMP-9) may be associated with lower activity of MMP-9 and lower level of MSDx complex-1 (see FIG. 1). Conversely, a lower level of TIMP-1 may be associated with a higher level of MMP-9 activity and level of MSDx complex-1. Intermediate levels of TIMP-1 correlate with intermediate levels of MSDx complex-1.

Without wishing to limit the present invention to any theory or mechanism, it is believed that the data suggests that MSDx complex-1 may be generated by cell transmigration into tissues. For example, MSDx complex-1 may be generated by proteolytic activity of leukocytes (or other cell types) crossing the blood vessel wall and tissue barriers in order to enter the target organ.

Further, a disease that is characterized by movement of leukocytes (or other cell types) into tissues can be monitored by the measurement of MSDx complex-1. Diseases that may be monitored or detected by measurement of MSDx complex-1 include but are not limited to: autoimmune diseases, e.g., multiple sclerosis, rheumatoid arthritis, lupus, sjogren's syndrome, thyroiditis, uveitis, Crohn's disease, ulcerative colitis, psoriasis, type 1 diabetes mellitus, autoimmune Addison's disease, autoimmune hepatitis, celiac disease, pemphigous, chronic inflammatory demyelinating polyneuropathy, acute disseminated encephalomyelopathy, sarcoidosis, dermatomyositis and behcet's disease; neurological diseases, e.g., stroke, concussion, chronic traumatic encephalopathy, neuromyelitis optica, transverse myelitis, intractable epilepsy and CNS infections; Parkinson's disease; primary tumor growth, metastasis of tumors; etc.

Window into the Brain

Various debris antigens may be found in recirculating phagocytes in the peripheral blood. Such debris antigens may be used to detect (or monitor) neurodegenerative or neuroinflammatory diseases (e.g., diseases as described above). Antigens include but are not limited to: a Tau protein (or fragment thereof), a Tau protein or fragment thereof comprising a phosphorylated residue (e.g., a phosphorylated serine reside, a phosphorylated threonine reside; e.g., serine 214, serine 235, serine 262, serine 356, serine 396, serine 404, serine 413, serine 46, serine 515, serine 516, serine 519, serine 531, serine 552, serine 610, serine 622, serine 641, serine 713, serine 721, serine 726, serine 730, serine 739, threonine 181, threonine 205, threonine 470, threonine 492, threonine 498, threonine 522, threonine 529, threonine 534, threonine 548; a protein or a fragment thereof selected from the group consisting of UCHL-1, neuromelanin, neuroglobin, valosin-containing protein, brain hexokinase, hippocalcin-1, nestin, synaptotagmin, myelin associated glycoprotein, transketolase, NS1 associated protein 1, major vault protein, synaptojanin, enolase, alpha synuclein, glial fibrillary acidic protein, S-100 protein, Neu-N, 26S proteasome subunit 9, annexin A2, annexin A3, annexin A5, annexin A6, annexin A11, ubiquitin activating enzyme ZE1, ubiquitin B precursor, vimentin, glyceraldehyde-3-phosphate dehydrogenase, 13-3-3 protein, 14-3-3 protein (e.g., zeta isoform), NOGO-A.

Another debris antigen that may be found in recirculating phagocytes in the peripheral blood may include neuromelanin (or a fragment thereof). Neuromelanin may be used to detect Parkinson's disease. For example, neuromelanin may be detected in the debris of degenerated dopaminergic neurons (by recirculating phagocytes).

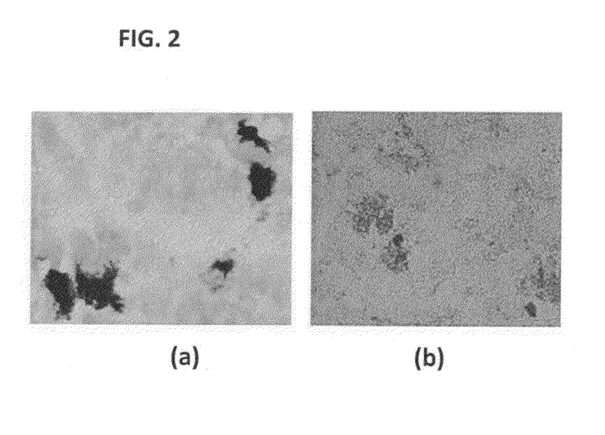

Neuromelanin can be measured in several ways, e.g., via the binding of labeled melanin selective peptides (e.g., 4B4 peptide (SEQ ID NO:1A), e.g., biotinylated 4B4 peptide, a control peptide P601G (DGASYSWMYGA (SEQ ID NO:2A)) may be used as a control); the binding of monoclonal or polyclonal antibodies to melanin; measurement of metal binding to melanin; measurement of the semiconductor properties of melanin; measurement of the fluorescence properties of melanin; and extraction of melanin from recirculating phagocytes and subsequent quantification of melanin, it's components or adducts (both natural or synthetic); physical methods such as gas chromatography, liquid chromatography or mass spectrometry; and combinations of these methods. As shown in FIG. 2, the 4B4 peptide of sequence YERKFWHGRH (SEQ ID NO:1A) binds to neuromelanin granules in the dopaminergic neurons of the human substantia nigra.

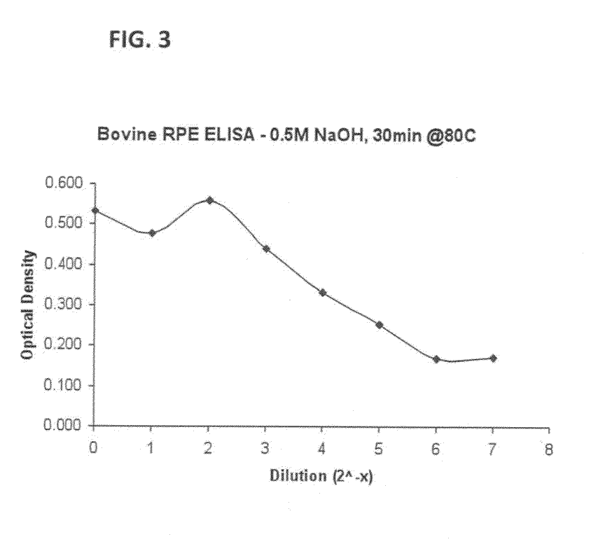

As an example of a means of measurement of melanin within cells, FIG. 3 shows the binding of the 4B4 peptide. Extracts of retinal pigment epithelium were immobilized on ELISA plates in two-fold dilution series of the extract and incubated with biotinylated 4B4 peptide. Unbound peptide was washed off and bound peptide was detected with streptavidin-HRP. In comparison, PBMCs from healthy human subjects show little binding of 4B4 peptide.

In some embodiments, more than one biomarker is detected in the sample(s). In some embodiments, the biomarker(s) is a neural-derived biomarker. However, the biomarker(s) is not limited to neural-derived biomarkers. In some embodiments, one or more biomarkers are detected in the sample, wherein the biomarkers are neural-derived, non-neural-derived biomarkers, or a combination thereof.

As used herein, the term “peripheral” refers to anything outside of brain tissue. For example, a peripheral phagocyte may be obtained from cerebrospinal fluid (CSF). Phagocytes may include monocytes, macrophages, and/or lymphocytes. Such circulating phagocytes may be found in tissues, cells, and/or fluids in the body, for example in blood, peripheral blood mononuclear cells (PBMCs), synovial fluid, cerebrospinal fluid (CSF), central nervous system tissues, synovial fluid, cystic fluid, lymph fluid, ascites, pleural effusion, interstitial fluid, ocular fluids, vitreal fluid, urine the like, or a combination thereof. In some embodiments, the biomarker is an intracellular component. For example, the biomarker may be obtained from within a macrophage. In some embodiments, the macrophage sample is permeabilized. In some embodiments, the macrophage is lysed via various means, e.g., hypotonic solution treatment, detergent solution treatment, mechanical stress, etc.

### Detectable Diseases and Biomarkers for Detection

As previously discussed, the present invention features the detection of and/or the monitoring of various diseases via detection/measurement of various biomarkers in recirculating phagocytes.

For example, in some embodiments, the a disease detected or monitored includes (but is not limited to): autoimmune diseases, e.g., multiple sclerosis, rheumatoid arthritis, lupus, sjogren's syndrome, thyroiditis, uveitis, Crohn's disease, ulcerative colitis, psoriasis, type 1 diabetes mellitus, autoimmune addison's disease, autoimmune hepatitis, celiac disease, pemphigous, chronic inflammatory demyelinating polyneuropathy, acute disseminated encephalomyelopathy, sarcoidosis, dermatomyositis and behcet's disease; neurological diseases, e.g., stroke, concussion, chronic traumatic encephalopathy, neuromyelitis optica, transverse myelitis, intractable epilepsy and CNS infections; Parkinson's disease; primary tumor growth, metastasis of tumors; etc.

In some embodiments, a biomarker detected or measured in recirculating phagocytes includes (but is not limited to): Neuromelanin (or a fragment thereof); a Tau protein (or fragment thereof), a Tau protein or fragment thereof comprising a phosphorylated residue (e.g., a phosphorylated serine reside, a phosphorylated threonine reside; e.g., serine 214, serine 235, serine 262, serine 356, serine 396, serine 404, serine 413, serine 46, serine 515, serine 516, serine 519, serine 531, serine 552, serine 610, serine 622, serine 641, serine 713, serine 721, serine 726, serine 730, serine 739, threonine 181, threonine 205, threonine 470, threonine 492, threonine 498, threonine 522, threonine 529, threonine 534, threonine 548; a protein or a fragment thereof selected from the group consisting of UCHL-1, neuromelanin, neuroglobin, valosin-containing protein, brain hexokinase, hippocalcin-1, nestin, synaptotagmin, myelin associated glycoprotein, transketolase, NS1 associated protein 1, major vault protein, synaptojanin, enolase, alpha synuclein, glial fibrillary acidic protein, S-100 protein, Neu-N, 26S proteasome subunit 9, annexin A2, annexin A3, annexin A5, annexin A6, annexin A11, ubiquitin activating enzyme ZE1, ubiquitin B precursor, vimentin, glyceraldehyde-3-phosphate dehydrogenase, 13-3-3 protein, 14-3-3 protein (e.g., zeta isoform), NOGO-A, Ubiquitin carboxy-terminal hydrolase L1 (UCHL1) also known as PARK5 protein; neuronal-specific protein gene product 9.5; SwissProt P09936; proteolipid protein; myelin oligodendrocyte glycoprotein.

Table A shows non-limiting examples of biomarkers that may be associated with disease states (e.g., degenerative disease states) with various organs. For example, the present invention may be used to detect a diabetes condition by detecting somatostatin in a similar manner as described herein, e.g., similar to methods for detecting neuromelanin for Parkinson's disease.

TABLE A

| Damaged Organ | Associated Markers |
|---|---|
| Joints (eg Rheumatoid | Citrulinated proteins |
| Arthritis) | Carbamylated proteins |
| | Articular cartilage degradation products |
| Central Nervous System | Neuromelanin (or a fragment thereof); a Tau |
| (eg multiple sclerosis, | protein (or fragment thereof), a Tau protein or |
| Parkinsons disease, | fragment thereof comprising a phosphorylated |
| Alzheimer disease etc). | residue (e.g., a phosphorylated serine reside, a |
| | phosphorylated threonine reside; e.g., serine |
| | 214, serine 235, serine 262, serine 356, serine |
| | 396, serine 404, serine 413, serine 46, serine |
| | 515, serine 516, serine 519, serine 531, serine |
| | 552, serine 610, serine 622, serine 641, serine |
| | 713, serine 721, serine 726, serine 730, serine |
| | 739, threonine 181, threonine 205, threonine |
| | 470, threonine 492, threonine 498, threonine |
| | 522, threonine 529, threonine 534, threonine |
| | 548; a protein or a fragment thereof selected |
| | from the group consisting of UCHL-1, |
| | neuromelanin, neuroglobin, valosin-containing |
| | protein, brain hexokinase, hippocalcin-1, nestin, |
| | synaptotagmin, myelin associated glycoprotein, |
| | transketolase, NS1 associated protein 1, major |
| | vault protein, synaptojanin, enolase, alpha |
| | synuclein, glial fibrillary acidic protein, S-100 |
| | protein, Neu-N, 265 proteasome subunit 9, |
| | annexin A2, annexin A3, annexin A5, annexin |
| | A6, annexin A11, ubiquitin activating enzyme |
| | ZE1, ubiquitin B precursor, vimentin, |
| | glyceraldehyde-3-phosphate dehydrogenase, |
| | 13-3-3 protein, 14-3-3 protein (e.g. zeta |
| | isoform), NOGO-A, Ubiquitin carboxy-terminal |
| | hydrolase LI (UCHL1) also known as PARK5 |
| | protein; neuronal-specific protein gene product |
| | 9.5; SwissProt P09936; proteolipid protein; |
| | myelin oligodendrocyte glycoprotein. |
| Thyroid (eg Graves | Thyroglobulin |
| disease, Hashimotos | Thyroid peroxidase |
| thyroiditis) | |
| Retina (eg macular | Rhodopsin |
| degeneration, retinitis | |
| pigmentosa) | |
| Pancreatic islets | Insulin, Glucagon, somatostatin, |
| (Diabetes) | pancreatic polypeptide |
| Inflammatory bowel | Microbial glycans |
| disease | |
| Lung | Surfactant proteins A-D |
| Sjogrens Syndrome | Salivary proline rich proteins |
| severe traumatic brain | all-spectrin breakdown products (SBDPs) |
| injury patients | |

### Methods of Detecting a Neurological Condition Via Analysis of Circulating Phagocytes

Multiple Sclerosis (MS) is predominantly a disease of women of northern European origin and afflicts up to three million people worldwide. In the United States it is estimated that 400,000 people are affected. It is thought to be an autoimmune disorder and typically strikes young adults, causing a wide variety of symptoms that are often mistaken for other diseases. These symptoms stem from disruption of the central nervous system (CNS) and may include blurred or double vision; weakness in the arms or legs; changes or difficulties in balance, coordination and gait; bladder and/or bowel dysfunction; and emotional disturbances. Each patient may present a little differently and there may have been episodes in the past that were barely noticed by the patient at the time. It is difficult to firmly diagnose MS, especially if there has been only one symptomatic episode. This leaves patients and their doctors waiting months or years for a relapse to confirm that the symptoms are due to MS.

MS is a demyelinating disease, where myelin, the insulating layer on nerve fibers, is destroyed in the CNS, which consists of the brain, optic nerves, and spinal column. There is an accompanying inflammatory response and the blood brain barrier (BBB) is breached. Axon damage can occur and the optic nerve is commonly affected. Myelin damage makes it more difficult for nerves to transmit impulses, leading to symptoms of MS. The diagnostic McDonald Criteria (1) were revised in 2005 to include magnetic resonance imaging (MRI) criteria of different types of lesions of the brain and spinal cord in the diagnosis of MS. Prognosis is difficult to determine, and many brain lesions do not necessarily correlate with severity of disease. There are medications available to alleviate some symptoms and a few others to modify and hopefully delay the onset or severity of relapses of MS.

The most common form of MS is relapsing-remitting multiple sclerosis (RRMS), which is characterized by symptomatic episodes separated in time, with partial or complete recovery of an apparently normal state between relapses. It often converts to secondary progressive MS after several years, where there is a steady worsening of symptoms. A minority of patients have Primary Progressive MS which presents as a continuous slow worsening of the disease state. An even smaller minority of patients is diagnosed with Progressive-Relapsing MS, where in contrast to RRMS, there is a continuous worsening of their condition between acute episodes. A first episode is referred to as Clinically Isolated Syndrome (CIS) pending a more certain diagnosis of MS corresponding to clinical signs and/or brain lesions visualized by MRI, or possibly a spinal tap to check for immunoglobulin oligoclonal bands (OCB) in the cerebral spinal fluid (CSF). None of these diagnostic methods is 100% specific. (2). Its drawbacks include the expense and the fact that a patient must wait one to three months between scans to determine if new lesions have formed during the intervening period. There is a clear need for identification of a biomarker or set of biomarkers that indicate presence and/or severity of disease for MS patients. A simple blood test would be ideal for diagnosing MS, however at this time, no commercial blood test exists.

Early diagnosis of MS is thought to be increasingly important, as much of the damage occurs early in the disease process. The earlier the diagnosis, the earlier disease-modifying treatment can begin and progression of the disease and associated disability can hopefully be slowed.

The present invention features a method of detecting multiple sclerosis or a risk of multiple sclerosis. The present invention also features methods of determining the status of a disease or condition or monitoring disease activity and drug efficacy. The method comprises detecting a multiple sclerosis-associated biomarker, e.g., an antigen, wherein detecting an elevated level of such multiple sclerosis-associated biomarker indicates the presence of multiple sclerosis or a risk of multiple sclerosis. In some embodiments, the antigens detected in accordance with the present invention includes, for example, Ubiquitin carboxy-terminal hydrolase L1 (UCHL1) also known as PARK5 protein; neuronal-specific protein gene product 9.5; SwissProt P09936.

### Inflammatory Conditions

The present invention features a method of detecting an inflammatory condition. The method comprises providing a first sample (e.g., a fluid sample) that contains a peripheral (e.g., circulating) phagocyte, and detecting one or more biomarkers, e.g., an antigen, inside a phagocyte of said fluid sample, wherein the biomarker is associated with an inflammatory condition. The sample may be provided from a mammal (e.g., a patient, a mouse, a rat, etc.). The fluid obtained does not necessarily directly come into contact with the inflamed tissue being detected. For example, there may be a barrier between the fluid and the source of the biomarker. In other words, the fluid obtained may have once directly come into contact with the inflamed tissue, but at the time that it is being extracted in accordance with the present invention, it is being separated from the inflamed tissue by a barrier.

The method may further comprise comparing the level of the biomarker in the first sample with a level of the biomarker in a second sample (e.g., a fluid sample). The second sample may be a control sample. In some embodiments, the second sample is a fluid sample from outside of a brain tissue comprising a peripheral (e.g., circulating phagocyte), The second sample may be provided from a mammal (e.g., a patient, a mouse, a rat, etc.). The second sample may have been collected prior to the first fluid sample.

As used herein, the term “peripheral” refers to anything outside of brain tissue. For example, a peripheral phagocyte may be obtained from cerebrospinal fluid (CSF). Phagocytes may include monocytes, macrophages, and/or lymphocytes. Such circulating phagocytes may be found in tissues, cells, and/or fluids in the body, for example in blood, peripheral blood mononuclear cells (PBMCs), synovial fluid, cerebrospinal fluid (CSF), central nervous system tissues, synovial fluid, cystic fluid, lymph fluid, ascites, pleural effusion, interstitial fluid, ocular fluids, vitreal fluid, urine the like, or a combination thereof. In some embodiments, the biomarker is an intracellular component. For example, the biomarker may be obtained from within a macrophage. In some embodiments, the macrophage sample is permeabilized. In some embodiments, the macrophage is lysed via various means, e.g., hypotonic solution treatment, detergent solution treatment, mechanical stress, etc.

In some embodiments, the sample is a plasma sample.

Also, as used herein, “a fluid that does not directly come into contact with the inflamed tissue” is a fluid that is separated from the inflamed tissue by at least one barrier, e.g., a tissue membrane, a layer of cells, etc.

In some embodiments, one or more biomarkers are detected in the collected fluid sample. For example, a pattern of biomarkers may be detected in the sample. Detecting the biomarker or biomarkers indicates the presence of the inflammatory condition or a risk of the inflammatory condition. In some embodiments, detecting an increased level of the biomarker or biomarkers as compared to the level of the biomarker or biomarkers of a control sample indicates the presence of the inflammatory condition or a risk thereof. A control sample is discussed below. In some embodiments, detecting a decreased level of the biomarker or biomarkers as compared to the level of the biomarker or biomarkers of a control sample indicates the presence of the inflammatory condition or a risk thereof.

In some embodiments, the inflammation condition that may be monitored or detected includes Rheumatoid Arthritis, Systemic Lupus Erythematosis, Shogren's Syndrome, and the like.

In some embodiments, the biomarker(s) is a neural-derived biomarker. However, the biomarker(s) is not limited to neural-derived biomarkers. In some embodiments, one or more biomarkers are detected in the sample, wherein the biomarkers are neural-derived, non-neural-derived biomarkers, or a combination thereof.

The biomarker(s) may be detected using a variety of methods. Methods may include an immunoassay, a histological assay, a flow cytometry assay, the like, or a combination thereof. For example, in some embodiments, the step of detecting the biomarker(s) in the sample may comprise introducing an antibody to the sample, wherein the antibody binds to the biomarker or is specific for the biomarker.

In some embodiments, this method of detecting an inflammatory condition is used in combination with one or more different methods for detecting the inflammatory disease. In some embodiments, this method is used to differentiate between one or more inflammatory conditions.

### Neurological Conditions

The present invention also features a method of detecting a neurological condition. The method comprises providing a first sample (e.g., a fluid sample) that comprises a peripheral (e.g., circulating) phagocyte. The first sample may be derived from outside of a brain tissue. The method further comprises detecting one or more biomarkers, e.g., an antigen, inside a phagocyte of said sample, wherein the biomarker is associated with a neurological condition (e.g., a neurological condition-associated protein). The sample may be provided from a mammal (e.g., a patient, a mouse, a rat, etc.). Detecting the neurological condition-associated protein indicates the presence of the neurological condition or a risk of the neurological condition.

The sample (e.g., fluid) obtained does not necessarily directly come into contact with the inflamed tissue being detected. For example, in some embodiments, there may be a barrier between the fluid and the source of the biomarker. In other words, the fluid obtained may have once directly come into contact with the inflamed tissue, but at the time that it is being extracted in accordance with the present invention, it is being separated from the inflamed tissue by a barrier.

The method may further comprise comparing the level of the biomarker in the first sample with a level of the biomarker in a second sample (e.g., a fluid sample). The second sample may be a control sample. In some embodiments, the second sample is a fluid sample from outside of a brain tissue comprising a peripheral (e.g., circulating phagocyte), The second sample may be provided from a mammal (e.g., a patient, a mouse, a rat, etc.). The second sample may have been collected prior to the first fluid sample.

In some embodiments, detecting an increased level of the neurological condition-associated protein as compared to the level of the neurological condition-associated protein of a control sample indicates the presence of the neurological condition or a risk thereof. In some embodiments, detecting a decreased level of the neurological condition-associated protein as compared to the level of the neurological condition-associated protein of a control sample indicates the presence of the neurological condition or a risk thereof. A control sample is discussed below.

In some embodiments, the neurological condition that may be monitored or detected includes Alzheimer's Disease, Parkinson's Disease, Neuromyelitis Optica, transverse myelitis, Acute and chronic Stroke, Traumatic Brain Injury, and the like.

In some embodiments, the neurological condition-associated protein is derived from a brain source. In some embodiments, the neurological condition-associated protein is derived from a non-brain source. In some embodiments, one or more neurological condition-associated proteins is derived from a brain source, a non-brain source, or a combination thereof.

The neurological condition-associated protein may be present in a circulating phagocyte. Phagocytes may include monocytes, macrophages, and/or lymphocytes. Such circulating phagocytes may be found in tissues, cells, and/or fluids in the body, for example in blood, peripheral blood mononuclear cells (PBMCs), cerebrospinal fluid (CSF), central nervous system tissues, synovial fluid, cystic fluid, lymph fluid, ascites, pleural effusion, interstitial fluid, ocular fluids, vitreal fluid, urine, the like, or a combination thereof. In some embodiments, the neurological condition-associated protein is an intracellular component. For example, the neurological condition-associated protein may be obtained from within a macrophage. In some embodiments, the macrophage sample is permeabilized. In some embodiments, the macrophage is lysed via various means, e.g., hypotonic solution treatment, detergent solution treatment, mechanical stress, etc.

The neurological condition-associated protein may be detected using a variety of methods. Methods may include an immunoassay, a histological assay, a flow cytometry assay, the like, or a combination thereof. In some embodiments, the step of detecting the neurological condition-associated protein in the sample may comprise introducing an antibody to the sample, wherein the antibody binds to the protein or is specific for the protein.

In some embodiments, this method is used in combination with one or more different methods for detecting the neurological condition. In some embodiments, this method is used to differentiate between one or more neurological conditions.

### Multiple Sclerosis

The present invention also features methods of detecting multiple sclerosis or a risk of multiple sclerosis. In some embodiments, the methods of the present invention may allow for monitoring, detecting and/or predicting a relapse or a remission of multiple sclerosis. In some embodiments, the method of detecting multiple sclerosis is used in combination with one or more methods of detecting multiple sclerosis. For example, the present methods may be used in conjunction with other modalities to monitor, detect or predicting a relapse or a remission of multiple sclerosis.

The method of detecting multiple sclerosis comprises (1) providing a first sample (e.g., a fluid sample) that comprises a peripheral (e.g., circulating) phagocyte. The first sample may be derived from outside of a brain tissue, and (2) detecting a multiple sclerosis-associated biomarker in the phagocyte. The sample may be provided from a mammal (e.g., a patient, a mouse, a rat, etc.). In some embodiments, one or more multiple sclerosis-associated biomarkers is detected in the sample. The multiple sclerosis-associated biomarkers are associated with multiple sclerosis.

The method may further comprise comparing the level of the biomarker in the first sample with a level of the biomarker in a second sample (e.g., a fluid sample). The second sample may be a control sample. In some embodiments, the second sample is a fluid sample from outside of a brain tissue comprising a peripheral (e.g., circulating phagocyte), The second sample may be provided from a mammal (e.g., a patient, a mouse, a rat, etc.). The second sample may have been collected prior to the first fluid sample. Detecting the multiple sclerosis-associated biomarker may indicate the presence of multiple sclerosis or a risk of multiple sclerosis.

In some embodiments, detecting an increased level of the multiple sclerosis-associated biomarker as compared to the level of the multiple sclerosis-associated biomarker of a control sample indicates the presence of multiple sclerosis or a risk thereof. In some embodiments, detecting a decreased level of the multiple sclerosis-associated biomarker as compared to the level of the multiple sclerosis-associated biomarker of a control sample indicates the presence of multiple sclerosis or a risk thereof. In some embodiments, detecting an increased level of one class of multiple sclerosis-associated biomarker and a decreased of another class of multiple sclerosis-associated biomarker as compared to the respective level of the multiple sclerosis-associated biomarker of a control sample indicates the presence of multiple sclerosis or a risk thereof.

In some embodiments, the sample is obtained from the mammal immediately following a relapse (e.g., exacerbation of symptoms) before a drug (e.g., a steroid) treatment has begun. In some embodiments, the sample is obtained from the mammal before a relapse. In some embodiments, the sample is obtained during the course of the drug (e.g., steroid) treatment.

The multiple sclerosis-associated biomarker may be present in a circulating phagocyte. Phagocytes may include monocytes, macrophages, and/or lymphocytes. For example, macrophages are a type of monocyte and are phagocytic cells important in both specific cell-mediated immunity and non-specific innate immunity. Circulating phagocytes may be found in tissues, cells, and/or fluids in the body, for example in blood, peripheral blood mononuclear cells (PBMCs), cerebrospinal fluid (CSF), central nervous system tissues, synovial fluid, cystic fluid, lymph fluid, ascites, pleural effusion, interstitial fluid, ocular fluids, vitreal fluid, urine, the like, or a combination thereof. In some embodiments, the neurological condition-associated protein is an intracellular component. For example, the neurological condition-associated protein may be obtained from within a macrophage. In some embodiments, the macrophage sample is permeabilized.

As used herein, a mammal includes a human, a mouse, a rat, a llama, a rabbit, a dog, a primate, a guinea pig, a cat a hamster, a pig, a chicken, a goat, a horse, or a cow.

In some embodiments, the multiple sclerosis-associated biomarker is a Tau protein (or a fragment thereof) or a Tau protein (or fragment thereof) comprising a phosphorylated residue (e.g., a phosphorylated serine reside, a phosphorylated threonine reside). In some embodiments, the phosphorylated residue is serine 214, serine 235, serine 262, serine 356, serine 396, serine 404, serine 413, serine 46, serine 515, serine 516, serine 519, serine 531, serine 552, serine 610, serine 622, serine 641, serine 713, serine 721, serine 726, serine 730, serine 739, threonine 181, threonine 205, threonine 470, threonine 492, threonine 498, threonine 522, threonine 529, threonine 534, threonine 548, the like, or a combination thereof.

In some embodiments, phosphorylation of Tau can decrease its solubility. In some embodiments, the method of detecting multiple sclerosis comprises detecting a level of insoluble Tau protein in the sample. In some embodiments, an increased level of insoluble Tau protein as compared to a control level of insoluble Tau protein is indicative of multiple sclerosis or a risk thereof.

In some embodiments, the multiple sclerosis-associated biomarker is a protein or a fragment thereof selected from the group consisting of neuroglobin, valosin-containing protein, brain hexokinase, hippocalcin-1, nestin, synaptotagmin, myelin associated glycoprotein, myelin basic protein, myelin oligodendrocyte glycoprotein, myelin proteolipid protein, transketolase, NS1 associated protein 1, major vault protein, synaptojanin, enolase, alpha synuclein, glial fibrillary acidic protein, S-100 proteinNeu-N, 26S proteasome subunit 9, annexin A2, annexin A3, annexin A5, annexin A6, annexin A11, ubiquitin activating enzyme ZE1, ubiquitin B precursor, vimentin, glyceraldehyde-3-phosphate dehydrogenase, 13-3-3 protein, 14-4-4 protein, neurofilament heavy chain and neurofilament light chain.

The multiple sclerosis-associated biomarker (e.g., Tau protein or fragment thereof) may be of various lengths. For example, in some embodiments, the multiple sclerosis-associated biomarker consists of between about 5 to 20 amino acids. In some embodiments, the multiple sclerosis-associated biomarker consists of about 20 to 40 amino acids. In some embodiments, the multiple sclerosis-associated biomarker consists of about 40 to 80 amino acids. In some embodiments, the multiple sclerosis-associated biomarker consists of about 80 to 150 amino acids. In some embodiments, the multiple sclerosis-associated biomarker consists of about 150 to 200 amino acids. In some embodiments, the multiple sclerosis-associated biomarker consists of about 200 to 300 amino acids. In some embodiments, the multiple sclerosis-associated biomarker consists of about 300 to 400 amino acids. In some embodiments, the multiple sclerosis-associated biomarker consists of about 400 to 500 amino acids. In some embodiments, the multiple sclerosis-associated biomarker consists of about 500 to 600 amino acids.

The multiple sclerosis-associated biomarker (e.g., Tau protein or fragment thereof) may comprise various regions of the full-length protein. For example, in some embodiments, the multiple sclerosis-associated biomarker comprises the amino-terminus (e.g., N-terminus, NH2-terminus, N-terminal end, amine-terminus). The amino-terminus refers to the amino acid at the end of a protein or polypeptide that has a free amine group (—NH2). In some embodiments, the multiple-sclerosis associated biomarker consists of about the first 15 amino acids. In some embodiments, the multiple-sclerosis associated biomarker consists of about the first 25 amino acids. In some embodiments, the multiple-sclerosis associated biomarker consists of about the first 50 amino acids. In some embodiments, the multiple-sclerosis associated biomarker consists of about the first 75 amino acids. In some embodiments, the multiple-sclerosis associated biomarker consists of about the first 100 amino acids. In some embodiments, the multiple-sclerosis associated biomarker consists of about the first 125 amino acids.

In some embodiments, the multiple-sclerosis associated biomarker or fragment thereof comprises the carboxy-terminus (e.g., C-terminus, COOH-terminus, C-terminal end, carboxyl-terminus). The carboxy-terminus refers to the amino acid at the end of a protein or polypeptide that has a free carboxylic acid group (—COOH). In some embodiments, the multiple-sclerosis associated biomarker consists of about the last 100 amino acids.

### Monitoring Disease Activity and Drug Efficacy

The present invention also features methods of determining the status of a disease or condition (e.g., a neurological condition, an inflammatory condition, multiple sclerosis, etc.) or determining the status of drug efficacy. The present invention may also feature methods of monitoring disease activity and drug efficacy. For example, biomarkers can be used to detect a disease or condition and the biomarkers may be used to determine severity of the disease or condition (e.g. relapse, remission, etc.).

In some embodiments, the method comprises providing a sample (e.g., a fluid, a brain tissue), the sample comprising a circulating phagocyte. The sample may be derived from a mammal (e.g., a patient, a mouse, a rat, etc.). A biomarker or level thereof associated with a disease or condition (e.g., a multiple sclerosis-associated biomarker) may be detected in the sample (e.g., in the phagocyte) and compared to the level or presence of the biomarker in control samples. In some embodiments, the biomarker detected may be compared to the level or presence of the biomarker in a second sample, the second sample having been collected prior to the first sample. By comparing the level or presence of the biomarker in the sample to either a control sample or a patient's previous sample, disease activity may be determined. A biomarker may include but is not limited to Tau or a fragment thereof.

In some embodiments, the monitoring of disease activity may be used to determine drug efficacy. In some embodiments, the monitoring of disease activity may be used to determine drug failure and/or breakthrough disease. In some embodiments, the monitoring of disease activity may be used to determine patient compliance with drug therapy. In some embodiments, the monitoring of disease activity may be used to determine therapeutic non-responders. In some embodiments, the monitoring of disease activity may be used to aid drug development.

In some embodiments, the present invention features a method of monitoring disease activity of a neurological condition, the method comprises obtaining from a mammal a first fluid sample from outside of a brain tissue of the mammal, the first fluid sample comprises a first circulating phagocyte; detecting a level of a biomarker associated with the neurological condition in the first sample; and comparing the level of the biomarker in the first sample with a level of the biomarker in a second sample, the second sample being either a control sample or a second fluid sample from outside of a brain tissue of the mammal, the second fluid sample comprising a second circulating phagocyte, the second fluid sample having been taken prior to the first fluid sample.

Table 1 shows the amino acid sequence of full-length human Tau protein.

TABLE 1

| Length | | | | |
|---|---|---|---|---|
| (# of | | | | |
| amino | MW | | Amino | SEQ |
| Acids) | (Daltons) | Amino Acid Sequence | Acids | ID NO |
| 758 AA | 78,878 Da | MAEPRQEFEV MEDHAGTYGL GDRKDQGGYT | 1-758 | 1 |
| [This is | [This is the | MHQDQEGDTD AGLKESPLQT PTEDGSEEPG | | |
| the length | MW of the | SETSDAKSTP TAEDVTAPLV DEGAPGKQAA | | |
| of the | unprocessed | AQPHTEIPEG TTAEEAGIGD TPSLEDEAAG | | |
| unprocessed | precursor] | HVTQEPESGK VVQEGFLRFP GPPGLSHQLM | | |
| precursor] | | SGMPGAPLLP EGPREATRQP SGTGPEDTEG | | |
| | | GRHAPELLKH QLLGDLHQEG PPLKGAGGKF | | |
| | | RPGSKEEVDE DRDVDESSPQ DSPPSKASPA | | |
| | | QDGRPPQTAA REATSIPGFP AEGATPLPVD | | |
| | | FISKVSTEIP ASEPDGPSVG RAKGQDAPLE | | |
| | | FTFHVEITPN VQKEQAHSEE HLGRAAFPGA | | |
| | | PGEGPEARGI SLGEDTKEAD LPEPSEKQPA | | |
| | | AAPRGKPVSR VTQLKARMVS KSKDGTGSDD | | |
| | | KKAKTSTRSS ANTLKNRPCL SPKLPTPGSS | | |
| | | DPLIQPSSPA VCPEPPSSPN HVSSVTSRTG | | |
| | | SSGAKEMKLN GADGKTKTAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | NNVKSKIGST ENLKHQPGGG KVQIINKKLD | | |
| | | LSNVQSKCGS KDNIKHVPGG GSVQIVYKPV | | |
| | | DLSKVISKQG SLGNIHUNIG GGQVEVKSEK | | |
| | | LDFKDRWQSN IGSLDNITHV PGGGNKKIET | | |
| | | HNITFRENAK AKTDHGAEIV YKSPVVSGDI | | |
| | | SPRHLSNVSS TGSIDMVDSP QLAILADEVS | | |
| | | ASLAKQGL | | |

Table 2 shows examples of some of the possible multiple sclerosis-associated biomarkers (e.g., Tau protein or a fragment there).

TABLE 2

| Length (# | | | | SEQ |
|---|---|---|---|---|
| of Amino | MW | | Amino | ID |
| Acids) | (Daltons) | Amino Acid Sequence | Acids | NO |
| 10 | 1235.4 | MAEPRQEFEV | 1-10 | 2 |
| 20 | 2310.58 | MAEPRQEFEV MEDHAGTYGL | 1-20 | 3 |
| 30 | 3388.69 | MAEPRQEFEV MEDHAGTYGL GDRKDQGGYT | 1-30 | 4 |
| 40 | 4545.84 | MAEPRQEFEV MEDHAGTYGL GDRKDQGGYT | 1-40 | 5 |
| | | MHQDQEGDTD | | |
| 50 | 5571.02 | MAEPRQEFEV MEDHAGTYGL GDRKDQGGYT | 1-50 | 6 |
| | | MHQDQEGDTD AGLKESPLQT | | |
| 60 | 6570 | MAEPRQEFEV MEDHAGTYGL GDRKDQGGYT | 1-60 | 7 |
| | | MHQDQEGDTD AGLKESPLQT PTEDGSEEPG | | |
| 70 | 7574.04 | MAEPRQEFEV MEDHAGTYGL GDRKDQGGYT | 1-70 | 8 |
| | | MHQDQEGDTD AGLKESPLQT PTEDGSEEPG | | |
| | | SETSDAKSTP | | |
| 80 | 8571.17 | MAEPRQEFEV MEDHAGTYGL GDRKDQGGYT | 1-80 | 9 |
| | | MHQDQEGDTD AGLKESPLQT PTEDGSEEPG | | |
| | | SETSDAKSTP TAEDVTAPLV | | |
| 90 | 9496.14 | MAEPRQEFEV MEDHAGTYGL GDRKDQGGYT | 1-90 | 10 |
| | | MHQDQEGDTD AGLKESPLQT PTEDGSEEPG | | |
| | | SETSDAKSTP TAEDVTAPLV DEGAPGKQAA | | |
| 100 | 10556.29 | MAEPRQEFEV MEDHAGTYGL GDRKDQGGYT | 1-100 | 11 |
| | | MHQDQEGDTD AGLKESPLQT PTEDGSEEPG | | |
| | | SETSDAKSTP TAEDVTAPLV DEGAPGKQAA | | |
| | | AQPHTEIPEG | | |
| 110 | 11501.26 | MAEPRQEFEV MEDHAGTYGL GDRKDQGGYT | 2-110 | 12 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD | | |
| 120 | 12472.27 | MAEPRQEFEV MEDHAGTYGL GDRKDQGGYT | 1-120 | 13 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| 130 | 13565.44 | MAEPRQEFEV MEDHAGTYGL GDRKDQGGYT | 1-130 | 14 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK | | |
| 140 | 14720.77 | MAEPRQEFEV MEDHAGTYGL GDRKDQGGYT | 1-140 | 15 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP | | |
| 150 | 15738.98 | MAEPRQEFEV MEDHAGTYGL GDRKDQGGYT | 1-150 | 16 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| 160 | 16660.12 | MAEPRQEFEV MEDHAGTYGL GDRKDQGGYT | 1-160 | 17 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP | | |
| 170 | 17782.35 | MAEPRQEFEV MEDHAGTYGL GDRKDQGGYT | 1-170 | 18 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP | | |
| 180 | 18713.25 | MAEPRQEFEV MEDHAGTYGL GDRKDQGGYT | 1-180 | 19 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| 190 | 19822.53 | | 1-190 | 20 |
| 200 | 20879.8 | MAEPRQEFEV MEDHAGTYGL GDRKDQGGYT | 1-200 | 21 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG | | |
| 210 | 21847.88 | MAEPRQEFEV MEDHAGTYGL GDRKDQGGYT | 1-210 | 22 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| 220 | 23050.05 | MAEPRQEFEV MEDHAGTYGL GDRKDQGGYT | 1-220 | 23 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE | | |
| 230 | 24062.07 | MAEPRQEFEV MEDHAGTYGL GDRKDQGGYT | 1-230 | 24 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ | | |
| 240 | 25070.18 | MAEPRQEFEV MEDHAGTYGL GDRKDQGGYT | 1-240 | 25 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| 250 | 26125.36 | MAEPRQEFEV MEDHAGTYGL GDRKDQGGYT | 1-250 | 26 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA | | |
| 260 | 27062.44 | MAEPRQEFEV MEDHAGTYGL GDRKDQGGYT | 1-260 | 27 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP | | |
| 270 | 28148.74 | MAEPRQEFEV MEDHAGTYGL GDRKDQGGYT | 1-270 | 28 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| 280 | 29145.79 | MAEPRQEFEV MEDHAGTYGL GDRKDQGGYT | 1-280 | 29 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP | | |
| 290 | 30141.88 | MAEPRQEFEV MEDHAGTYGL GDRKDQGGYT | 1-290 | 30 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG | | |
| 300 | 31313.22 | MAEPRQEFEV MEDHAGTYGL GDRKDQGGYT | 1-300 | 31 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| 310 | 32422.47 | MAEPRQEFEV MEDHAGTYGL GDRKDQGGYT | 1-310 | 32 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN | | |
| 320 | 33510.63 | MAEPRQEFEV MEDHAGTYGL GDRKDQGGYT | 1-320 | 33 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE | | |
| 330 | 34449.64 | MAEPRQEFEV MEDHAGTYGL GDRKDQGGYT | 1-330 | 34 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| 340 | 35433.69 | MAEPRQEFEV MEDHAGTYGL GDRKDQGGYT | 1-340 | 35 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP | | |
| 350 | 36529.87 | MAEPRQEFEV MEDHAGTYGL GDRKDQGGYT | 1-350 | 36 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD | | |
| 360 | 37535.06 | MAEPRQEFEV MEDHAGTYGL GDRKDQGGYT | 1-360 | 37 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| 370 | 38611.37 | MAEPRQEFEV MEDHAGTYGL GDRKDQGGYT | 1-370 | 38 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR | | |
| 380 | 39701.64 | MAEPRQEFEV MEDHAGTYGL GDRKDQGGYT | 1-380 | 39 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS | | |
| 390 | 40719.73 | MAEPRQEFEV MEDHAGTYGL GDRKDQGGYT | 1-390 | 40 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| 400 | 41806.98 | MAEPRQEFEV MEDHAGTYGL GDRKDQGGYT | 1-400 | 41 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS | | |
| 410 | 42900.36 | MAEPRQEFEV MEDHAGTYGL GDRKDQGGYT | 1-410 | 42 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL | | |
| 420 | 43892.47 | MAEPRQEFEV MEDHAGTYGL GDRKDQGGYT | 1-420 | 43 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| 430 | 44857.59 | MAEPRQEFEV MEDHAGTYGL GDRKDQGGYT | 1-430 | 44 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA | | |
| 440 | 45867.71 | MAEPRQEFEV MEDHAGTYGL GDRKDQGGYT | 1-440 | 45 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK | | |
| 450 | 46828.72 | MAEPRQEFEV MEDHAGTYGL GDRKDQGGYT | 1-450 | 46 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| 460 | 47858.97 | MAEPRQEFEV MEDHAGTYGL GDRKDQGGYT | 1-460 | 47 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK | | |
| 470 | 48822.13 | MAEPRQEFEV MEDHAGTYGL GDRKDQGGYT | 1-470 | 48 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT | | |
| 480 | 49775.15 | MAEPRQEFEV MEDHAGTYGL GDRKDQGGYT | 1-480 | 49 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| 490 | 50804.12 | MAEPRQEFEV MEDHAGTYGL GDRKDQGGYT | 1-490 | 50 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA | | |
| 500 | 51782.51 | MAEPRQEFEV MEDHAGTYGL GDRKDQGGYT | 1-500 | 51 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP | | |
| 410 | 52807.56 | MAEPRQEFEV MEDHAGTYGL GDRKDQGGYT | 1-510 | 52 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| 520 | 53701.53 | MAEPRQEFEV MEDHAGTYGL GDRKDQGGYT | 1-520 | 53 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP | | |
| 530 | 54735.74 | MAEPRQEFEV MEDHAGTYGL GDRKDQGGYT | 1-530 | 54 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP | | |
| 540 | 55677.7 | MAEPRQEFEV MEDHAGTYGL GDRKDQGGYT | 1-540 | 55 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| 550 | 56295.63 | MAEPRQEFEV MEDHAGTYGL GDRKDQGGYT | 1-550 | 56 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP | | |
| 560 | 57337.84 | MAEPRQEFEV MEDHAGTYGL GDRKDQGGYT | 1-560 | 57 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL | | |
| 570 | 58388.1 | MAEPRQEFEV MEDHAGTYGL GDRKDQGGYT | 1-570 | 58 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| 580 | 59431.32 | MAEPRQEFEV MEDHAGTYGL GDRKDQGGYT | 1-580 | 59 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST | | |
| 590 | 60449.41 | MAEPRQEFEV MEDHAGTYGL GDRKDQGGYT | 1-590 | 60 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG | | |
| 600 | 61629.85 | MAEPRQEFEV MEDHAGTYGL GDRKDQGGYT | 1-600 | 61 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| 610 | 62633.98 | MAEPRQEFEV MEDHAGTYGL GDRKDQGGYT | 1-610 | 62 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS | | |
| 620 | 63680.16 | MAEPRQEFEV MEDHAGTYGL GDRKDQGGYT | 1-620 | 63 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG | | |
| 630 | 64751.44 | MAEPRQEFEV MEDHAGTYGL GDRKDQGGYT | 1-630 | 64 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| 640 | 65770.63 | MAEPRQEFEV MEDHAGTYGL GDRKDQGGYT | 1-640 | 65 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG | | |
| 650 | 66811.8 | MAEPRQEFEV MEDHAGTYGL GDRKDQGGYT | 1-650 | 66 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG | | |
| 660 | 67853.95 | MAEPRQEFEV MEDHAGTYGL GDRKDQGGYT | 1-660 | 67 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| 670 | 69071.34 | MAEPRQEFEV MEDHAGTYGL GDRKDQGGYT | 1-670 | 68 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK | | |
| 680 | 70121.52 | MAEPRQEFEV MEDHAGTYGL GDRKDQGGYT | 1-680 | 69 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV | | |
| 690 | 71103.62 | MAEPRQEFEV MEDHAGTYGL GDRKDQGGYT | 1-690 | 70 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| 70072329.04 | | MAEPRQEFEV MEDHAGTYGL GDRKDQGGYT | 1-700 | 71 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK | | |
| 710 | 73351.17 | MAEPRQEFEV MEDHAGTYGL GDRKDQGGYT | 1-710 | 72 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK AKTDHGAEIV | | |
| 720 | 74385.31 | MAEPRQEFEV MEDHAGTYGL GDRKDQGGYT | 1-720 | 73 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK AKTDHGAEIV YKSPVVSGDT | | |
| 730 | 75450.47 | MAEPRQEFEV MEDHAGTYGL GDRKDQGGYT | 1-730 | 74 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK AKTDHGAEIV YKSPVVSGDT | | |
| | | SPRHLSNVSS | | |
| 740 | 76804.91 | MAEPRQEFEV MEDHAGTYGL GDRKDQGGYT | 1-740 | 75 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK AKTDHGAEIV YKSPVVSGDT | | |
| | | SPRHLSNVSS TGSIDMVDSP | | |
| 750 | 78109.39 | MAEPRQEFEV MEDHAGTYGL GDRKDQGGYT | 1-750 | 76 |
| | | MHQDQEGDTD AGLKESPLQT PTEDGSEEPG | | |
| | | SETSDAKSTP TAEDVTAPLV DEGAPGKQAA | | |
| | | AQPHTEIPEG TTAEEAGIGD TPSLEDEAAG | | |
| | | HVTQEPESGK VVQEGFLREP GPPGLSHQLM | | |
| | | SGMPGAPLLP EGPREATRQP SGTGPEDTEG | | |
| | | GRHAPELLKH QLLGDLHQEG PPLKGAGGKE | | |
| | | RPGSKEEVDE DRDVDESSPQ DSPPSKASPA | | |
| | | QDGRPPQTAA REATSIPGFP AEGAIPLPVD | | |
| | | FLSKVSTEIP ASEPDGPSVG RAKGQDAPLE | | |
| | | FTFHVEITPN VQKEQAHSEE HLGRAAFPGA | | |
| | | PGEGPEARGP SLGEDTKEAD LPEPSEKQPA | | |
| | | AAPRGKPVSR VPQLKARMVS KSKDGTGSDD | | |
| | | KKAKTSTRSS AKTLKNRPCL SPKLPTPGSS | | |
| | | DPLIQPSSPA VCPEPPSSPK HVSSVTSRTG | | |
| | | SSGAKEMKLK GADGKTKIAT PRGAAPPGQK | | |
| | | GQANATRIPA KTPPAPKTPP SSGEPPKSGD | | |
| | | RSGYSSPGSP GTPGSRSRTP SLPTPPTREP | | |
| | | KKVAVVRTPP KSPSSAKSRL QTAPVPMPDL | | |
| | | KNVKSKIGST ENLKHQPGGG KVQIINKKLD | | |
| | | LSNVQSKCGS KDNIKHVPGG GSVQIVYKPV | | |
| | | DLSKVTSKCG SLGNIHHKPG GGQVEVKSEK | | |
| | | LDFKDRVQSK IGSLDNITHV PGGGNKKIET | | |
| | | HKLTFRENAK AKTDHGAEIV YKSPVVSGDT | | |
| | | SPRHLSNVSS TGSIDMVDSP QLATLADEVS | | |
| 10 | 1815.06 | QLATLADEVS ASLAKQGL | 749-758 | 77 |
| 20 | 2818.17 | TGSIDMVDSP QLATLADEVS ASLAKQGL | 739-758 | 78 |
| 30 | 2992.33 | SS TGSIDMVDSP QLATLADEVS | 729-758 | 79 |
| | | ASLAKQGL | | |
| 40 | 4099.53 | DT SPRHLSNVSS TGSIDMVDSP | 719-758 | 80 |
| | | QLATLADEVS ASLAKQGL | | |

In some embodiments, the step of detecting the multiple sclerosis-associated biomarker in the sample may comprise introducing an antibody to the sample, wherein the antibody binds to the multiple sclerosis-associated biomarker.

In some embodiments, the step of detecting the multiple sclerosis-associated biomarker in the sample comprises subjecting the sample to a western blot, an enzyme-linked immunosorbent assay (ELISA), a lateral flow assay, a radioimmunoassay, an immunohistochemistry assay, a bioluminescent assay, a chemiluminescent assay, a mass spectrometry assay, a flow cytometry assay (e.g., florescence-activated cell sorting (FACS)), or a combination thereof and the like. Such assays are well known in the art.

In some embodiments, the step of detecting the multiple sclerosis-associated biomarker further comprises contacting the sample with an antibody that binds to the multiple sclerosis-associated biomarker and detecting an antibody-biomarker complex. The step of detecting an antibody-biomarker complex may comprise subjecting the sample to a micro array, western blot, an enzyme-linked immunosorbent assay (ELISA), a lateral flow assay, a radioimmunoassay, an immunohistochemistry assay, a bioluminescent assay, a chemiluminescent assay, a flow cytometry assay (e.g., florescence-activated cell sorting (FACS)), or a combination thereof and the like. In some embodiments, detecting the antibody-biomarker complex indicates the presence of multiple sclerosis or a risk of multiple sclerosis.

As described above, in some embodiments, the step of detecting the multiple sclerosis-associated biomarker may comprise subjecting the sample florescence-activated cell sorting (FACS). Fluorescence-activated cell sorting (FACS) is a type of flow cytometry that sorts a mixture of biological cells, one at a time, into separate containers based upon the specific light scattering and fluorescent characteristics of each cell. It provides quantitative recording of fluorescent signals from individual cells as well as physical separation of cells of particular interest. Generally, a current of a rapidly flowing stream of liquid carries a suspension of cells through a nozzle. The flow is selected such that there is a large separation between cells relative to their diameter. Vibrations at the tip of the nozzle cause the stream of cells to break into individual droplets, and the system is adjusted so that there is a low probability of more than one cell being in a droplet. A monochromatic laser beam illuminates the droplets, which are electronically monitored by fluorescent detectors. The droplets that emit the proper fluorescent wavelengths are electrically charged between deflection plates in order to be sorted into collection tubes.

As described above, in some embodiments, the step of detecting the multiple sclerosis-associated biomarker may comprise subjecting the sample to an enzyme-linked immunosorbent assay (ELISA). ELISA is an assay used to detect the presence of an antibody or a biomarker in a sample. Generally, in ELISA, a sample containing an unknown amount of biomarker, e.g., an antigen, is affixed/immobilized to a surface (e.g., a polystyrene microtiter plate). Then, an antibody that binds to the antigen of interest is washed over the surface so that it can bind the antigen and form an antibody/antigen complex. In some cases, this antibody is covalently linked to an enzyme. In some cases, the antibody is not covalently linked to an enzyme but can be detected by a secondary antibody that is linked to an enzyme. In the final step, a substance (e.g., substrate) that the enzyme is capable of converting to a detectable visible signal (e.g., color signal) is added to the reaction. Thus, if the antibody/antigen complex is present, the substrate will be converted to the detectable visible signal, and then amount of antigen in the sample can be measured.

As mentioned above, in some embodiments, an antibody is used to detect the presence of the multiple sclerosis-associated biomarker. The multiple sclerosis-associated biomarker may be detected with a variety of antibodies. In some embodiments, the antibody is a monoclonal or a polyclonal antibody. In some embodiments, the antibody is a humanized antibody. In some embodiments, the antibody is a chimera. In some embodiments, the antibody is derived from a human, a mouse, a rat, a llama, a rabbit, a dog, a primate, a guinea pig, a cat, a hamster, a pig, a chicken, a goat, a horse, or a cow. In some embodiments, the antibody is synthetic. In some embodiments, the antibody is a recombinant antibody.

Frequently, antibodies are labelled either covalently or non-covalently by combining the antibody with a second substance that provides for detectable signal. A wide variety of labels and conjugation techniques are known in the art and are reported extensively in both the scientific and patent literature. Examples of labels include but are not limited to radioisotopes, enzymes, substrates, cofactors, inhibitors, fluorescers, chemiluminescers, magnetic particles, and the like. In some embodiments of the present invention, the antibody comprises a label.

In some embodiments, the present invention is used to detect the presence of multiple sclerosis. For example, a patient may present with symptoms of a demyelinating disease. A sample (e.g., derived from the patient) may be tested for an elevated level of a multiple sclerosis-associated biomarker. If, according to the present invention, the level of a multiple sclerosis-associated biomarker is elevated and the patient presents symptoms of a demyelinating disease, then the patient is diagnosed as having multiple sclerosis.

In some embodiments, the present invention is used to detect a risk of multiple sclerosis. For example, a patient may present with no symptoms of a demyelinating disease, but he or she wishes to be tested for a risk of multiple sclerosis. If, according to the present invention, the level of a multiple sclerosis-associated biomarker is elevated and the patient does not present symptoms of a demyelinating disease, then the patient is diagnosed as having a risk of multiple sclerosis.

As used herein, the term “elevated level” refers to a level that is higher than the normal level of the multiple sclerosis-associated biomarker (e.g., the level that would be detected in a person who does not have multiple sclerosis). To identify the level of the multiple sclerosis-associated biomarker that is the normal level, samples are pooled from about, for example, 500 patients (or an appropriate number of patients that would be statistically meaningful) who do not experience any symptoms of multiple sclerosis (or other demyelinating diseases) and who do not test positive for multiple sclerosis as detected by MRI. From those pooled samples, the average level of the multiple sclerosis-associated biomarker can be quantified and then defined as being the normal level of the multiple sclerosis-associated biomarker. If the normal level of the multiple sclerosis-associated biomarker is about zero, then an elevated level refers to any level that is greater than zero, for example, about 5 units, about 25 units, about 50 units, about 100 units, about 500 units, about 1000 units, about 10,000 units, about 100,000 units, about 1,000,000 units. In some embodiments, a unit may be an absorbance unit (e.g., from an ELISA), a percent positive (e.g., from a flow cytometry or FACS assay), or a fluorescence unit.

If the normal level of the multiple sclerosis-associated biomarker is some positive value (e.g., 5 units, 10 units, 50 units, 100 units, 500 units), then an elevated level refers to any level that is higher than the normal level. In some embodiments, an elevated level of the multiple sclerosis-associated biomarker may be a level that is about 10-20% higher than the normal level of the multiple sclerosis-associated biomarker. In some embodiments, an elevated level of the multiple sclerosis-associated biomarker may be a level that is about 20-30% higher than the normal level of the multiple sclerosis-associated biomarker. In some embodiments, an elevated level of the multiple sclerosis-associated biomarker may be a level that is about 30-40% higher than the normal level of the multiple sclerosis-associated biomarker. In some embodiments, an elevated level of the multiple sclerosis-associated biomarker may be a level that is about 40-50% higher than the normal level of the multiple sclerosis-associated biomarker. In some embodiments, an elevated level of the multiple sclerosis-associated biomarker may be a level that is about 50-60% higher than the normal level of the multiple sclerosis-associated biomarker. In some embodiments, an elevated level of the multiple sclerosis-associated biomarker may be a level that is about 60-70% higher than the normal level of the multiple sclerosis-associated biomarker. In some embodiments, an elevated level of the multiple sclerosis-associated biomarker may be a level that is about 70-80% higher than the normal level of the multiple sclerosis-associated biomarker. In some embodiments, an elevated level of the multiple sclerosis-associated biomarker may be a level that is about 80-90% higher than the normal level of the multiple sclerosis-associated biomarker. In some embodiments, an elevated level of the multiple sclerosis-associated biomarker may be a level that is about 90-100% higher than the normal level of the multiple sclerosis-associated biomarker. In some embodiments, an elevated level of the multiple sclerosis-associated biomarker may be a level that is about 1-2 fold higher than the normal level of the multiple sclerosis-associated biomarker. In some embodiments, an elevated level of the multiple sclerosis-associated biomarker may be a level that is about 2-3 fold higher than the normal level of the multiple sclerosis-associated biomarker. In some embodiments, an elevated level of the multiple sclerosis-associated biomarker may be a level that is about 3-4 fold higher than the normal level of the multiple sclerosis-associated biomarker. In some embodiments, an elevated level of the multiple sclerosis-associated biomarker may be a level that is about 4-5 fold higher than the normal level of the multiple sclerosis-associated biomarker. In some embodiments, an elevated level of the multiple sclerosis-associated biomarker may be a level that is about 5-10 fold higher than the normal level of the multiple sclerosis-associated biomarker. In some embodiments, an elevated level of the multiple sclerosis-associated biomarker may be a level that is about 10-20 fold higher than the normal level of the multiple sclerosis-associated biomarker. In some embodiments, an elevated level of the multiple sclerosis-associated biomarker may be a level that is about 20-50 fold higher than the normal level of the multiple sclerosis-associated biomarker.

The present invention also provides a method of monitoring the progression of multiple sclerosis and/or monitoring the treatment of multiple sclerosis. For example, in some embodiments, the present invention may be used to measure the level of the multiple sclerosis-associated biomarker in order to detect a change in the level (e.g., an increase in the level, a decrease in the level, a maintaining of the level). Without wishing to limit the present invention to any theory or mechanism, a change in the level of the multiple sclerosis-associated biomarker may correlate with a change in the patient's status (e.g., remission, progression, worsening). For example, a decrease in the level of the multiple sclerosis-associated biomarker may indicate the patient has entered or will enter a remission period. In some embodiments, the present invention may be used to monitor the level of the multiple sclerosis-associated biomarker in a patient while the patient is on a treatment regimen (e.g., a drug). Without wishing to limit the present invention to any theory or mechanism, a treatment regimen (e.g., a drug) that is effective at inhibiting the progression of multiple sclerosis and/or reducing the symptoms of multiple sclerosis may decrease the level of the multiple sclerosis-associated biomarker in the patient.

As mentioned above, in some embodiments, the method of the present invention for detecting multiple sclerosis is used in combination with one or more different methods for detecting multiple sclerosis. For example, in some cases, a combination of family history, a physical exam, and magnetic resonance imaging (MRI) findings are used to diagnose multiple sclerosis. Currently, MRI is the most sensitive radiographic technique for the imaging of multiple sclerosis. Multiple sclerosis plaques are commonly seen as round or void discrete lesions in the periventricular white matter. Other common locations for multiple sclerosis plaques include the corpus callosum, corona radiate, internal capsule, and centrum semiovale. In some embodiments, the present invention is used to measure a multiple sclerosis-associated biomarker, and the level of the multiple sclerosis-associated biomarker is correlated with a magnetic resonance imaging (MRI) measurement. Without wishing to limit the present invention to any theory or mechanism, it is believed that elevated levels of the multiple sclerosis-associated biomarker correlate with a MRI scan showing the presence of multiple sclerosis plaques in the brain.

The method of the present invention for detecting multiple sclerosis may be used in combination with one or more methods for detecting a different condition. For example, the method of the present invention may also help to distinguish multiple sclerosis from other diseases with similar clinical manifestations. For example, neuromyelitis optica (NMO), also known as Devic's syndrome, is a neurological disorder regarded as a severe variant of multiple sclerosis. The characteristic inflammatory demyelinating lesions of NMO selectively and repeatedly affect the optic nerves and the spinal cord, causing blindness and paralysis. A marker (e.g., aquaporin-4 antibodies) has been identified in serum and cerebrospinal fluid of patients with NMO, and the presence of a NMO marker (e.g., aquaporin-4 antibodies) may be used to distinguish NMO from multiple sclerosis. In some embodiments, the method of detecting the presence of multiple sclerosis or a risk of multiple sclerosis comprises detecting the presence or absence of at least two biomarkers (e.g., proteins, antigens, or the like) wherein at least one biomarker is detected in order to distinguish multiple sclerosis from a disease with similar clinical manifestations.

In some embodiments, the method of detecting the presence of multiple sclerosis or a risk of multiple sclerosis comprises detecting an elevated level of two or more multiple sclerosis-associated biomarkers. In some embodiments, the method of detecting the presence of multiple sclerosis or a risk of multiple sclerosis comprises detecting an elevated level of three or more multiple sclerosis-associated biomarkers.

The present invention also provides a method of diagnosing multiple sclerosis at an early stage of the disease before all clinical criteria are fulfilled, thus justifying early initiation of a multiple sclerosis-appropriate therapy.

The present invention also features a kit for detecting the status of a disease or condition (e.g., an inflammatory condition, a neurological condition, multiple sclerosis, etc.). The kit may comprise an antibody specific for a biomarker (e.g., a multiple sclerosis-associated biomarker), wherein the biomarker is a protein selected from the group consisting of Tau or a fragment thereof, phosphorylated Tau or a fragment thereof, neuroglobin, valosin-containing protein, brain hexokinase, hippocalcin-1, nestin, synaptotagmin, myelin associated glycoprotein, Myelin Basic Protein (MBP), Proteolipid Protein, Myelin Oligodendrocyte Glycoprotein, transketolase, NS1 associated protein 1, major vault protein, synaptojanin, enolase, alpha synuclein, glial fibrillary acidic protein, S-100 proteinNeu-N, 26S proteasome subunit 9, annexin A2, annexin A3, annexin A5, annexin A6, annexin A11, ubiquitin activating enzyme ZE1, ubiquitin B precursor, vimentin, glyceraldehyde-3-phosphate dehydrogenase, 13-3-3 protein, or fragments thereof. In some embodiments, the kit further comprises a means for detecting the binding of the antibody to the multiple sclerosis-associated biomarker. In some embodiments, the antibody is a monoclonal or a polyclonal antibody.

In some embodiments, the detection of perforin is used in combination with detection of a marker (e.g., MBP) in phagocyltes. For example, it has been surprisingly discovered that perforin levels can decline in CD16 cells as MBP levels increase.

### Kit for Detecting Multiple Sclerosis

The present invention also features a kit for detecting the presence of multiple sclerosis or a risk of multiple sclerosis in a circulating phagocyte sample derived from a mammal. The kit comprises an antibody that binds to a multiple sclerosis-associated biomarker. In some embodiments, the kit further comprises a means for detecting the binding of the antibody to the multiple sclerosis-associated biomarker/antigen in the sample (e.g., an antibody-antigen complex). In some embodiments, the detecting of an elevated level of an antibody-antigen complex indicates presence of multiple sclerosis or a risk of multiple sclerosis.

In some embodiments, the kit comprises an antibody, wherein the antibody is a monoclonal or a polyclonal antibody. In some embodiments, the antibody is derived from a human, a mouse, a rat, a llama, a rabbit, a dog, a primate, a guinea pig, a cat, a hamster, a pig, a chicken, a goat, a horse, or a cow. In some embodiments, the antibody is humanized. In some embodiments, the antibody is a chimera. In some embodiments, the antibody is specific for the multiple sclerosis-associated biomarker.

### Example 1

### Detecting Multiple Sclerosis in a Patient

The following example describes the detection of multiple sclerosis in a patient according to two methods disclosed in the present invention. A 24-year-old male patient presents to his primary care physician complaining of changes in vision, limb weakness, and extreme fatigue. He mentions his symptoms have been recurring over the last 3 months. The physician suspects the possibility of a tumor in the central nervous system (CNS) or a CNS disease, as well as multiple sclerosis. The physician obtains a blood sample to be sent to a diagnostic laboratory for multiple sclerosis testing, and also refers the patient to a neurologist.

The laboratory receives the patient's blood sample collected in a CPT tube. PBMCs are obtained from a BD Vacutainer™ CPT tube using a cell separation procedure. The cells are washed three times in 1×PBS and centrifuged in a horizontal rotor (swing-out head) for a minimum of 5 minutes at 1200 to 1500 RCF (Relative Centrifugal force). The supernatant is removed and the cells are resuspended in 1×PBS. After the final wash, extracts of the PBMCs are prepared by lysing with a hypotonic solution or other method. Then the lysate is subjected to assay involving an antibody that binds to Tau protein fragment comprising the phosphorylated serine residue Ser-404. The assay indicates that an elevated level of said Tau protein fragment is present in the PBMCs. The assay is the assay of example 2 or example 3. Thus, the results of the assay indicate that the patient has multiple sclerosis. The physician notifies the patient, who then begins treatment immediately.

The laboratory receives the patient's blood sample collected in a CPT tube. PBMCs are obtained from a BD Vacutainer™ CPT tube using a cell separation procedure. The cells are washed three times in 1×PBS and centrifuged in a horizontal rotor (swing-out head) for a minimum of 5 minutes at 1200 to 1500 RCF (Relative Centrifugal force). The supernatant is removed and the cells are resuspended in 1×PBS. The cells are then subjected to assay involving an antibody that binds to Tau protein fragment comprising the phosphorylated serine residue Ser-404. The assay is the assay of example 4.

### Example 2

### Direct ELISA Assay Protocol

The following example describes a direct ELISA assay used for detecting a multiple sclerosis-associated antigen in a sample. The protein concentration of the sample is determined using the BioRad™ (Bradford method) assay. MicroELISA plates are coated by addition of 100 μL of a 5-20 μg/mL solution of the sample, which is then incubated for 1 hour at 21° C. The wells are washed out with phosphate buffered solution (PBS) with 0.05% polysorbate (Tween 20™). The wells are then filled with 0.1 M glycine in PBS and incubated for 1 hour at 21° C. to block unoccupied binding sites. After rewashing the wells, 100 μL of an appropriate dilution of antibody in PBS-0.05% Tween™ 20 with 1% bovine serum albumin (BSA) is added and incubated for 1 hour at 21° C. The unbound antibody is then washed out with three exchanges of PBS-0.05% Tween™ 20. One hundred μL of an appropriately diluted horse radish peroxidase conjugated anti-immunoglobulin G (IgG) in PBS-0.05% Tween™ 20-1% BSA is then added to each well and incubated for 1 hour at 21° C. The wells are then washed twice with PBS-0.05% Tween™ 20 and finally with PBS. One hundred μL of soluble MTB substrate solution is added to each well and incubated for 30 minutes at 21° C. after which 100 μL of MTB stop reagent is added and the color intensity is measured at 450 nm using an ELISA plate reader.

Appropriate dilutions of the antigen and antibody are established by performing checkerboard titrations. Antigen concentrations in samples are interpolated from standard curves.

### Example 3

### Indirect ELISA Assay Protocol

The following example describes an indirect ELISA assay used for detecting a multiple sclerosis-associated antigen in various samples. This assay is constructed using polyclonal and monoclonal antibodies. ELISA wells are coated with polyclonal antibody at an appropriate concentration and the wells are washed and blocked as described above. Various dilutions of antigen containing samples are added to the wells and incubated for 1 hour at 21° C., after which the wells are washed 3 times with PBS-0.05% Tween™20. The monoclonal antibody is then added at an appropriate dilution in PBS-0.05% Tween™20-1% BSA and incubated for 1 hour at 21° C. The wells are then washed 3 times and an appropriately diluted horse radish peroxidase conjugated anti-mouse IgM in PBS-0.05% Tween™ 20-1% BSA is then added to each well and incubated for 1 hour at 21° C. The wells are then washed twice with PBS-0.05% Tween™ 20 and finally with PBS. One hundred μL of soluble MTB substrate solution is added to each well and incubated for 30 minutes at 21° C. after which 100 μL of MTB stop reagent is added and the color intensity is measured at 450 nm using an ELISA plate reader.

Appropriate dilutions of antigen and antibody are established by performing checkerboard titrations. Antigen concentrations in samples are interpolated from standard curves.

### Example 4

### Flow Cytometry Protocol

The following example describes a flow cytometry assay used for detecting a multiple sclerosis-associated antigen in various samples. PBMCs from multiple sclerosis (MS) subjects and control subjects are stained with fluorescent antibodies to the multiple sclerosis-associated antigen (e.g., Tau protein) and also with fluorescent labeled antibodies to cluster designation (CD) 3 T-lymphocyte marker or CD 19 B-Lymphocyte marker, CD68 intracellular monocyte marker and CD14 monocyte/macrophage cell surface marker. The labeled cells are analyzed by flow cytometry for qualitative or quantitative differences.

PBMCs are obtained from a BD Vacutainer™ CPT tube using a cell separation procedure. The cells are washed three times in 1×PBS and centrifuged in a horizontal rotor (swing-out head) for a minimum of 5 minutes at 1200 to 1500 RCF (Relative Centrifugal force). The supernatant is removed and the cells are resuspended in 1×PBS. After the final wash, the cells are resuspended to approximately 4.0 mL in 1×PBS. Approximately 50 μL of the cell suspension to be analyzed is transferred into tubes for double staining with selected antibody pairs. Ten μL of 40 mg/mL normal human IgG (Sigma-Aldrich) for a total of 400 μg is added to each tube to block FC binding. The appropriate cell surface monoclonal antibodies CD3 PE, CD19 PE or CD14 PE are added at this time and incubated for 20 minutes at room temperature.

One hundred μl of Dako Intrastain™ Reagent A (fixative) is added to each tube and then mixed gently with a vortex mixer to ensure that the cells are in suspension. Cells are incubated at room temperature for 15 minutes. Two mL of 1×PBS working solution is added to each test tube and mixed gently. The tubes are centrifuged at 300×g for 5 minutes. Supernatant is aspirated leaving about 50 μl of fluid. The fluid is mixed thoroughly to ensure that the cells are in suspension.

One hundred μL of Dako Intrastain™ Reagent B (permeabilization) is added to each tube. The appropriate amount of the antibody specific for the multiple sclerosis-associated antigen is added to the appropriate tubes. The tubes are mixed gently to ensure that the cells are in suspension and incubated at room temperature for 15-60 minutes. Two mL of 1×PBS working solution is added to each test tube and mixed gently. The tubes are centrifuged at 300×g for 5 minutes, and then the supernatant is aspirated, leaving approximately 50 μl of fluid. The fluid is mixed thoroughly to ensure that the cells are in suspension.

One hundred μL of Dako Intrastain™ Reagent B (permeabilization) is added to each tube. The appropriate volume of the 2nd step antibody conjugated to FITC (specific to the multiple sclerosis-associated antigen) is added to the appropriate tubes. The tubes are mixed gently to ensure that the cells are in suspension and incubated at room temperature for 15-60 minutes. To each tube, 2.0 mLs of 1×PBS working solution is added. The tubes are mixed gently then centrifuged at 300×g for 5 minutes. The supernatant is aspirated, leaving approximately 50 μl of fluid. The tubes are mixed thoroughly to ensure that the cells are in suspension.

The pellet is resuspended in an appropriate volume of fluid for flow cytometry analysis. The sample is analyzed on a flow cytometer within 24-48 hours. For analysis, the gate is on the monocyte population and the data is collected in list mode. Qualitative and or quantitative differences are determined between normal and MS patients using the analysis software. Optimization steps include varying incubation time with antibodies, fixation time and permeabilization time.

Methods for Measuring High Molecular Weight Complexes of Fibrinogen with Fibronectin and Fibulin-1 (MSDX Complex-1)

It has been surprisingly discovered that the expression of a protein complex (e.g. an aggregate, a complex) termed “MSDX Complex-1” is elevated in multiple sclerosis patients as compared to healthy controls. MSDX Complex-1 is a high molecule weight complex comprising fibrinogen, fibronectin, and fibulin-1. MSDX Complex-1 alone or in combination with other markers may be useful as an indicator of multiple sclerosis or other diseases or conditions, for example for an inflammatory condition, a neurodegenerative disease or condition, cancer, stroke, or other diseases. MSDx complex-1 alone or in combination with one or more other biomarkers may help monitor disease activity (e.g., relapse, remission, etc.). Monitoring disease activity may be useful for detecting a response (e.g., positive response, negative response, lack of response) to a therapy, for detecting patient compliance with a therapy, or for providing useful clinical information for disease management.

The present invention features methods for measuring high molecular weight complexes of fibrinogen with fibronectin and fibulin-1 (“MSDx Complex-1”) and applications thereof. The methods may be used to monitor disease activity and therapeutic efficacy in diseases or conditions that have an inflammatory component, for example autoimmune diseases, neurodegenerative diseases, cancers and metabolic diseases such as type 2 diabetes mellitus. The present invention is not limited to the aforementioned diseases and conditions or the aforementioned applications.

The present invention features methods for measuring high molecular weight complexes of MSDX Complex-1, e.g., fibrinogen with fibronectin and fibulin-1, in a sample. As used herein, the term “MSDx Complex-1” refers to a high molecular weight complex of fibrinogen, fibronectin, and fibulin-1. The detection of MSDX Complex-1 may be used for a variety of purposes, for example for detecting a disease or condition, for monitoring a disease or condition, for monitoring a therapy, etc.

A circulating high molecular weight protein complex has been found to bind certain small peptides selectively. For example, by sephacryl S200 gel filtration chromatography, the binding activity was found in a broad peak of 400,000-900,000 kD. This peak was collected and shown by LC/MS, after in solution protease digestion, to consist of Fibrinogen, Fibronectin and Fibulin-1. The present invention features a unique competitive ELISA assay format to measure the amount of MSDx Complex-1 in a sample, e.g., plasma, by its ability to compete with an anti-peptide antibody for binding of the labeled peptide (e.g., biotinylated peptide). In some embodiments, the method comprises introducing a labeled peptide and an anti-peptide antibody to a sample to create an antibody-sample mixture. The anti-peptide antibody can bind to at least the labeled peptide and MSDX Complex-1. The labeled peptide comprises a label molecule (e.g., biotin). The label molecule is not limited to biotin but may include any appropriate label. Labels are well known to one of ordinary skill in the art.

In some embodiments, the method further comprises providing a well (e.g., ELISA well) coated with a “well antibody”. The well antibody is specific for a complex of labeled peptide and anti-peptide antibody. The method further comprises introducing the antibody-sample mixture to the well and introducing a substrate to the antibody-sample mixture in the well. The label molecule of the labeled peptide and the substrate interact to provide a signal. The level of the signal is compared to a control. If the level of the signal is higher than that of the control, then MSDX Complex-1 is not detected. If the level of the signal is lower than that of the control then MSDX Complex-1 is detected.

In some embodiments, the labeled peptide is or comprises SEQ ID NO:3A. In some embodiments, the labeled peptide is or comprises SEQ ID NO: 4A. In some embodiments, the labeled peptide is or comprises SEQ ID NO: 5A.

In some embodiments, the label of the labeled peptide is located at the C-terminus, the N-terminus or at both termini. In some embodiments, the labeled peptide is between about 15 to 50 amino acids in length, e.g., 24 amino acids, between about 15 to 40 amino acids, between about 15 to 30 amino acids, between about 20 to 30 amino acids, etc. In some embodiments, the labeled peptide has a pl of about 6.1. In some embodiments, the labeled peptide has a pl between about 6 and 7.0, between about 5.5 and 6.5, between about 5.8 and 6.4, etc. In some embodiments, the labeled peptide has a net charge of about −0.1 at pH 7.0. In some embodiments, the labeled peptide comprises an epitope tag disposed at the C-terminus, the N-terminus, or at both termini.

The present invention also features a method of detecting MSDX Complex-1 comprising introducing a first antibody to a sample to create an antibody-sample mixture, wherein the first antibody is specific for one of fibrinogen, fibronectin, or fibulin-1. The first antibody comprises a label molecule (e.g., HRP). A well (e.g., ELISA well) is provided. The well is coated with a second antibody, wherein the second antibody is specific for one of fibrinogen, fibronectin, or fibulin-1. In some embodiments, the method further comprises introducing the antibody-sample mixture to the well and introducing a substrate to the antibody-sample mixture in the well. The label molecule and the substrate interact to provide a signal (e.g., a chemiluminescent signal, a fluorescent signal, a colorimetric signal, a potentiometric signal, an amperometric signal, or a combination thereof). When the signal is detected then MSDX Complex-1 is detected.

In some embodiments, the first antibody is an anti-fibulin-1 antibody and the second antibody is an anti-fibrinogen antibody. In some embodiments, the first antibody is an anti-fibronectin antibody and the second antibody is an anti-fibrinogen antibody. In some embodiments, the first antibody is an anti-fibrinogen antibody and the second antibody is an anti-fibrinogen antibody. In some embodiments, the first antibody is an anti-fibulin-1 antibody and the second antibody is an anti-fibronectin antibody. In some embodiments, the first antibody is an anti-fibronectin antibody and the second antibody is an anti-fibronectin antibody. In some embodiments, the first antibody is an anti-fibrinogen antibody and the second antibody is an anti-fibronectin antibody. In some embodiments, the first antibody is an anti-fibulin-1 antibody and the second antibody is an anti-fibulin-1 antibody. In some embodiments, the first antibody is an anti-fibronectin antibody and the second antibody is an anti-fibulin-1 antibody. In some embodiments, the first antibody is an anti-fibrinogen antibody and the second antibody is an anti-fibulin-1 antibody.

In some embodiments, the method further comprises introducing a third antibody to the antibody-sample mixture prior to introduction to the well, the third antibody is specific for one of fibulin-1, fibronectin, or fibrinogen, wherein the third antibody has a different specificity than the first antibody. In some embodiments, the method further comprises introducing a fourth antibody to the antibody-sample mixture prior to introduction to the well, the third antibody is specific for one of fibulin-1, fibronectin, or fibrinogen, wherein the third antibody has a different specificity than the first antibody and a different specificity than the third antibody.

In some embodiments, the label molecule comprises an enzyme. In some embodiments, the enzyme comprises horseradish peroxidase.

In some embodiments, the first antibody is a rabbit antibody. The first antibody is not limited to rabbit and may be any other appropriate antibody (e.g., mouse, human, etc.). In some embodiments, the second antibody comprises an anti-rabbit antibody, e.g., a goat anti-rabbit antibody, a mouse anti-rabbit antibody, a human anti-rabbit antibody, etc.

As used herein, the term “about” refers to plus or minus 10% of the referenced number.

### Example 1A

The following example describes an example of a method of detecting MSDX Complex-1. Anti-Fibrinogen antibodies are immobilized onto an assay surface (e.g., ELISA well, glass slide, magnetic particle, antibody array matrix) and blocked using conventional methods. A biological fluid (e.g., serum, plasma, cerebrospinal fluid) is then contacted with the immobilized antibody and unbound material is washed off. Then antibodies to fibronectin and/or Fibulin-1 are contacted with the immobilized material and unbound antibodies are washed off. The bound antibodies are then detected with a labelled anti-immunoglobulin of the appropriate specificity to generate a measurable signal (the signal may be chemiluminescent, fluorescent, colorimetric, potentiometric, amperometric etc).

### Example 2A

The following example describes an example of a method of detecting MSDX Complex-1. In some embodiments, method is a competitive ELISA assay format. In some embodiments, the competitive ELISA assay used for detecting MDSX Complex-1 utilizes a labeled analyte and measures the ability of an unlabelled native analyte in a biological fluid to compete with the labeled analyte for binding to the antibody. In this assay the labeled analyte is bound by an unrelated binding protein that prevents it's binding to antibody and is washed off before the detection step. A standard curve to quantify binding by MDSX Complex-1 is generated by competition with “cold” peptide.

ELISA wells coated with goat anti-rabbit IgG(Fc) trap immune complexes formed between a rabbit anti-peptide antibody and biotinylated peptide. MSDX Complex-1 in added plasma competes with the rabbit anti-peptide antibody for binding to biotinylated peptide. The more MSDX Complex-1 that is present in the plasma the more biotinylated peptide it binds leaving less available to bind to antibody. Thus high levels of MSDX Complex-1 result in low optical density and vice versa.

In some embodiments, the peptide is labeled at the C-terminal with biotin or another detection agent. In some embodiments, the N-terminal of the peptide may be amine or amide. In some embodiments, the peptide is 24 amino acids long. In some embodiments, the peptide sequence is: CQYRCFQVITNGIGLNLFKDPVAD (SEQ ID NO: 3A). In some embodiments, the peptide has a pl of 6.1. In some embodiments, the peptide has a net charge of −0.1 at pH 7.0. In some embodiments, the peptide has an average hydrophilicity (Hopp & Woods method) of −0.3. In some embodiments, the peptide has a ratio of hydrophilic residues to total residues of 33%. In some embodiments, an epitope tag is attached to a terminus, e.g., the N-terminus, to enable the use of other capture antibodies, for example a polyHistidine tag (HisTag).

In some embodiments, any peptide sequence derived by conservative amino acid substitution rules such as the Dayhoff matrix and the like of SEQ ID NO: 3A may be used. In some embodiments, alternative peptides may be used.

In some embodiments, the peptide sequence is CSFKCYSVVTNGLGINVFKDPVAD (SEQ ID NO: 4A). In some embodiments, the peptide has a pl of 6.1. In some embodiments, the peptide has a net charge of −0.1 at pH 7.0. In some embodiments, the peptide has an average hydrophilicity (Hopp & Woods method) of −0.3. In some embodiments, the peptide has a ratio of hydrophilic residues to total residues of 33%.

In some embodiments, the peptide sequence is CQYRCFQIITNGIGLNLFKDPVAD (SEQ ID NO: 5A). In some embodiments, the peptide has a pl of 6.1. In some embodiments, the peptide has a net charge of −0.1 at pH 7.0. In some embodiments, the peptide has an average hydrophilicity (Hopp & Woods method) of −0.3. In some embodiments, the peptide has a ratio of hydrophilic residues to total residues of 33%.

The various peptides described bind selectively to a macromolecular complex consisting of Fibrinogen B, Fibronectin and Fibulin 1. The levels of this complex have surprisingly been found to be associated with neuroinflammatory diseases including multiple sclerosis. Addition of the peptide to plasma or serum causes the peptide to bind to the complex of Fibrinogen B, Fibronectin and Fibulin 1 effecting a transformation of matter that results in the formation of the aggrefatin complex.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method of detecting Parkinson's disease in a mammal, said method comprising:
(a) detecting a level of a biomarker associated with Parkinson's disease in a first sample from outside a brain tissue of the mammal, the first sample comprising a first circulating phagocyte; and
(b) comparing the level of the biomarker in the first sample with a level of the biomarker in a second sample, the second sample being either (i) a control sample or (ii) a second sample from outside of a brain tissue, the second sample comprising a second circulating phagocyte, the second sample being collected prior to the first fluid sample;
wherein if the level of the biomarker in the first sample is higher than that of the second sample then Parkinson's disease is detected.

2. The method of claim 1, wherein the sample is derived from blood, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, urine, the like, or a combination thereof.

3. The method of claim 1, wherein the biomarker associated with Parkinson's disease comprises neuromelanin or a fragment thereof.

4. The method of claim 1, wherein the circulating phagocyte includes a monocyte, a macrophage, a lymphocyte, or a combination thereof.

5. The method claim 3, wherein detecting the biomarker comprises subjecting the first sample and the second sample each to a peptide that binds to neuromelanin.

6. The method of claim 5, wherein the peptide that binds to neuromelanin comprises 4B4 (SEQ ID NO:1A).

7. A method of determining status of Parkinson's disease, the method comprises:
(a) detecting a level of a biomarker associated with Parkinson's disease in a first fluid sample from outside a brain tissue of the mammal, the first fluid sample comprising a first circulating phagocyte;
(b) comparing the level of the biomarker in the first sample with a level of the biomarker in a second sample, the second sample being either (i) a control sample or (ii) a second fluid sample from outside of a brain tissue, the second fluid sample comprising a second circulating phagocyte, the second fluid sample being collected prior to the first fluid sample.

8. The method of claim 7, wherein if the biomarker level in the first sample is the same as the level of the biomarker in the second sample or in the control sample, then Parkinson's disease activity is the same.

9. The method of claim 7, wherein if the biomarker level in the first sample is higher than the level of the biomarker in the second sample or in the control sample, then Parkinson's disease activity is increased in the first sample.

10. The method of claim 7, wherein if the biomarker level in the first sample is lower than the level of the biomarker in the second sample or in the control sample, then Parkinson's disease activity is decreased in the first sample.

11. The method of claim 7, wherein the sample is derived from blood, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, urine, the like, or a combination thereof.

12. The method of claim 7, wherein the circulating phagocyte includes a monocyte, a macrophage, a lymphocyte, or a combination thereof.

13. The method of claim 7, wherein the biomarker comprises neuromelanin or a fragment thereof.

14. A method of detecting neuromelanin, said method comprising:
(a) introducing a neuromelanin binding protein comprising a labeled 4B4 peptide (SEQ ID NO:1A) to a sample; and
(b) detecting the label on the 4B4 peptide.

15. The method of claim 14, wherein the sample comprises a circulating phagocyte.

16. The method of claim 14, wherein the sample comprises a circulating phagocyte derived from serum, plasma, peripheral blood mononuclear cells (PBMCs), cerebrospinal fluid (CSF), synovial fluid, cystic fluid, lymph fluid, ascites, pleural effusion, interstitial fluid, ocular fluids, vitreal fluid, or a combination thereof.

17. The method of claim 14, wherein the label comprises an enzyme.

18. The method of claim 14, wherein the label comprises biotin.

19. The method of claim 17, wherein the enzyme comprises horseradish peroxidase.
